(19) United States
(12) Patent Application Publication
(10) Pub. No.: US 2023/0103975 A1
(43) Pub. Date: Apr. 6, 2023

(54) INJECTOR FOR DELIVERING IMPLANTS

(71) Applicant: EyePoint Pharmaceuticals, Inc., Watertown, MA (US)

(72) Inventors: Michelle HOWARD-SPARKS, Newton, MA (US); Said SAIM, Wrentham, MA (US); Amelia SCHILLING, Boston, MA (US); Dario PAGGIARINO, Burlington, MA (US); Nathan ROLLINS, Boylston, MA (US); Evan Hunter WILLIAMS, Boston, MA (US); Phillip HALBERT, San Francisco, CA (US); Daniel P. SMITH, Portsmouth, RI (US); Michael W. METZ, Minneapolis, MN (US); Dustin GAIDOS, Milton, MA (US); Joshua MARTIN, Minneapolis, MN (US)

(21) Appl. No.: 17/958,924

(22) Filed: Oct. 3, 2022

Related U.S. Application Data
(60) Provisional application No. 63/251,799, filed on Oct. 4, 2021.
(60) Provisional application No. 63/359,281, filed on Jul. 8, 2022.

(51) Int. Cl. A61F 9/00

(52) U.S. Cl. CPC A61F 9/0017; A61B 17/3468

(57) ABSTRACT

An injector includes a push rod, a magazine tube, a gate, a cannula and an actuator. The magazine tube has a lumen extending from an inlet to an outlet thereof. The magazine tube slidingly receives at least one implant therein. The gate has a closed configuration in which it covers the outlet of the magazine tube and an open configuration in which it does not cover the outlet of the magazine tube. The cannula has a distal end configured to be inserted into an eye. A lumen of the cannula is in fluid communication with the lumen of the magazine tube when the gate is in the open configuration. Actuation of the actuator moves the gate from the closed configuration to the open configuration and causes translation of the pushrod through the magazine tube and the cannula.

### CROSS REFERENCE TO RELATED APPLICATIONS

This application claims the benefit of U.S. Pat. Appl. No. 63/251,799, filed Oct. 4, 2021, and U.S. Pat. Appl. No. 63/359,281, filed Jul. 8, 2022, the entire disclosures of which are hereby incorporated by reference herein for all purposes.

### FIELD OF THE INVENTION

The present disclosure relates in general to injectors and more particularly to an injector for delivering one or more implants.

### BACKGROUND OF THE INVENTION

A primary difficulty in treating diseases of the eye is introducing drugs or therapeutic agents into the eye and maintaining these drugs or agents at a therapeutically effective concentration in the eye for the necessary duration. Systemic administration may not be an ideal solution because, often, unacceptably high levels of systemic dosing are needed to achieve effective intraocular concentrations, with the increased incidence of unacceptable side effects of the drugs. Simple ocular instillation or application is not an acceptable alternative in many cases, because the drug may be quickly washed out by tear-action or pass from the eye into the general circulation. Suprachoroidal injections of drug solutions have also been performed, but again the drug availability is short-lived. Such methods make it difficult to maintain therapeutic levels of drug for adequate time periods. Efforts to address this problem have led to the development of drug delivery devices, or implants, which can be implanted into the eye such that a controlled amount of desired drug can be released constantly over a period of several days, or weeks, or even months.

Various sites exist in the eye for implantation of a drug delivery device or implant, such as the posterior segment of the eye, anterior or posterior chambers of the eye, or other areas of the eye including intraretinal, subretinal, intrachoroidal, suprachoroidal, intrascleral, episcieral, subconjunctival, intracorneal or epicorneal spaces. Wherever the desired location of implantation, typical methods of implantation all require relatively invasive surgical procedures, pose a risk of excessive trauma to the eye, and require excessive handling of the implant. For example, in a typical method for placement in the vitreous, an incision is made through the sclera, and the implant is inserted into and deposited at the desired location in the vitreous, using forceps or other like manual grasping device. Once deposited, the forceps (or grasping device) is removed, and the incision is sutured closed. Alternatively, an incision can be made through the sclera, a trocar can be advanced through the incision and then the implant can be delivered through the trocar. Similar methods can be employed to deliver implants to other locations, e.g., implantation in the anterior chamber of the eye through an incision in the cornea.

The drawbacks of such techniques for implant delivery are many. Extensive handling of the implant is necessitated in these techniques, creating a risk that the implant will be damaged or contaminated in the process. Many such implants are polymer-based and relatively fragile. If portions of such implants are damaged and broken off, the release profile and/or effective therapeutic dose delivered by the implant once placed will be significantly altered. In addition, achieving reproducible placement from patient to patient can be difficult using these methods. Also of import is that fact that such techniques may require an opening in the sclera large enough to require suturing. Thus, such techniques are typically performed in a surgical setting.

A more facile, convenient, less invasive, and/or less traumatic means for delivering implants into the eye is desirable.

### BRIEF SUMMARY OF THE INVENTION

According to a first embodiment hereof, the present disclosure provides an injector including a housing, a push rod disposed at least partially within the housing, a magazine tube disposed within the housing, a gate disposed within the housing, a cannula having a distal end that is disposed outside of the housing and is configured to be inserted into an eye, and an actuator. The magazine tube has an inlet, an outlet, and a lumen extending from the inlet to the outlet. The magazine tube is configured to slidingly receive at least one implant therein, and the push rod is configured to be slidingly received within the lumen of the magazine tube. The gate has a closed configuration in which it covers the outlet of the magazine tube and an open configuration in which it does not cover the outlet of the magazine tube. A lumen of the cannula is in fluid communication with the lumen of the magazine tube when the gate is in the open configuration. Actuation of the actuator moves the gate from the closed configuration to the open configuration and causes translation of the pushrod through the magazine tube and the cannula.

In an aspect of the first embodiment, and in combination with any other aspects herein, the disclosure provides that the injector further includes a safety cap that is configured to be removably coupled to the housing to cover the distal end of the cannula when the safety cap is coupled to the housing. The safety cap includes a tab that extends into a slot formed in the actuator when the safety cap is coupled to the housing to prevent actuation of the actuator.

In an aspect of the first embodiment, and in combination with any other aspects herein, the disclosure provides the housing has a generally tubular construction with an asymmetrical fin that includes a height that is greater than a height of the remaining length of the housing.

In an aspect of the first embodiment, and in combination with any other aspects herein, the disclosure provides that the distal end of the cannula is beveled.

In an aspect of the first embodiment, and in combination with any other aspects herein, the disclosure provides the push rod is attached to a shuttle body that is slidingly disposed within the housing and the shuttle body is coupled to a spring, the spring including a non-extended configuration and an extended configuration and being biased to the non-extended configuration. In an aspect of the first embodiment, and in combination with any other aspects herein, the disclosure provides that the spring is coiled in the non-extended configuration. In an aspect of the first embodiment, and in combination with any other aspects herein, the disclosure provides that the actuator in a non-deployed position holds the shuttle body such that the spring is in the extended configuration. In an aspect of the first embodiment, and in combination with any other aspects herein, the disclosure provides actuation of the actuator from the non-deployed position to a deployed position releases the shuttle body and permits the spring to resume the non-extended configuration. In an aspect of the first embodiment, and in combination with any other aspects herein, the disclosure provides when the actuator is in the non-deployed position, the actuator contacts and engages the shuttle body and when the actuator is in the deployed position, the actuator does not contact the shuttle body and is disengaged therefrom. In an aspect of the first embodiment, and in combination with any other aspects herein, the disclosure provides that the actuator is configured to rotate relative to the shuttle body to transition between the non-deployed position and the deployed position.

In an aspect of the first embodiment, and in combination with any other aspects herein, the disclosure provides that the magazine tube is configured to hold up to three implants and the injector is configured to deliver the three implants via a single actuation of the actuator.

In an aspect of the first embodiment, and in combination with any other aspects herein, the disclosure provides that the cannula and the magazine tube are coaxially aligned and a transition gap extends between the outlet of the magazine tube and an inlet of the cannula. In an aspect of the first embodiment, and in combination with any other aspects herein, the disclosure provides that a portion of the gate is disposed within the transition gap when the gate is in the closed configuration and the portion of the gate is not disposed within the transition gap when the gate is in the open configuration.

In an aspect of the first embodiment, and in combination with any other aspects herein, the disclosure provides that the housing includes a window formed thereon to permit visual feedback relating to the translation of the pushrod. In an aspect of the first embodiment, and in combination with any other aspects herein, the disclosure provides that the push rod is attached to a shuttle body that is slidingly disposed within the housing and an outer surface of the shuttle body includes a status indicator thereon for providing the visual feedback through the window.

In an aspect of the first embodiment, and in combination with any other aspects herein, the disclosure provides that the push rod is attached to a shuttle body that is slidingly disposed within the housing. A drag wire is attached to the shuttle body. The injector further includes a shuttle decelerator disposed within the housing, and the shuttle decelerator is configured to receive the drag wire within a sinusoidal path thereof. In an aspect of the first embodiment, and in combination with any other aspects herein, the disclosure provides the sinusoidal path of the shuttle decelerator is defined by a plurality of bosses and interaction between the drag wire and the plurality of bosses creates friction that slows down translation of the shuttle body and push rod attached thereto. In an aspect of the first embodiment, and in combination with any other aspects herein, the disclosure provides the drag wire is formed from stainless steel and the plurality of bosses are formed from a plastic material.

In an aspect of the first embodiment, and in combination with any other aspects herein, the disclosure provides that the push rod is formed from stainless steel.

In an aspect of the first embodiment, and in combination with any other aspects herein, the disclosure provides that the injector further includes a rotary damper disposed within the housing, the rotary damper being coupled to the push rod and configured to slow down the rate at which the push rod moves within the housing.

In an aspect of the first embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.035 in-lbs, and the injector has an injection speed of between 4 and 9 seconds.

In an aspect of the first embodiment, and in combination with any other aspects herein, the disclosure provides that the push rod is attached to a shuttle body that is slidingly disposed within the housing and the shuttle body is coupled to a spring, and wherein the spring has a spring constant for load of 0.4 pounds of force (0.4 lbf), the rotary damper has a damping torque of 0.026 in-lbs, and the injector has an injection speed of between 2.5 and 7.5 seconds.

In an aspect of the first embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.026 in-lbs, and the injector has an injection speed of between 1.5 and 6.5 seconds.

In an aspect of the first embodiment, and in combination with any other aspects herein, the disclosure provides a method of preventing or treating an ocular condition or disease of the eye in an eye in need thereof comprising using the injector of the first embodiment to administer an implant containing an active pharmaceutical ingredient (API). In some embodiments, the implant is administered to treat an anterior ocular condition. In other embodiments, it may be administered to treat a posterior ocular condition. In some embodiments, the implant is administered to prevent an anterior ocular condition. In other embodiments, it may be administered to prevent a posterior ocular condition.

In an aspect of the first embodiment, and in combination with any other aspects herein, the disclosure provides a method of treating treatment of chronic non-infectious uveitis affecting the posterior segment of the eye in an eye in need thereof comprising using the injector of the first embodiment to administer an implant containing fluocinolone acetonide. According to an embodiment, the implant is an intravitreal implant comprising about 0.18 mg fluocinolone acetonide. The implant may also comprise polyvinyl alcohol, silicone adhesive, a polyimide tube and may comprise water.

In an aspect of the first embodiment, and in combination with any other aspects herein, the disclosure provides a method of treating a retinal disease in an eye in need thereof comprising using the injector of the first embodiment to administer an implant comprising vorolanib. According to an embodiment, the implant is an intravitreal implant comprises about 400 µg to about 2800 µg vorolanib. The implant may also comprise polyvinyl alcohol.

According to a second embodiment hereof, the present disclosure provides a method of using an injector to deliver at least one implant into an eye. The injector is positioned near the eye. The injector includes a push rod, a magazine tube having an inlet, an outlet, and a lumen extending from the inlet to the outlet, the magazine tube having the at least one implant disposed therein, a gate in a closed configuration in which it covers the outlet of the magazine tube, a cannula, and an actuator. A distal end of the cannula is inserted into tissue of the eye. The actuator is actuated to deliver the at least one implant into the tissue of the eye. Actuation of the actuator moves the gate from the closed configuration to an open configuration in which the gate does not cover the outlet of the magazine tube and actuation of the actuator also causes translation of the pushrod through the magazine tube and the cannula to push the at least one implant.

In an aspect of the second embodiment, and in combination with any other aspects herein, the disclosure provides that the at least one implant includes exactly three implants.

In an aspect of the second embodiment, and in combination with any other aspects herein, the disclosure provides that the tissue of the eye includes a vitreous of the eye.

In an aspect of the second embodiment, and in combination with any other aspects herein, the disclosure provides a lumen of the cannula is in fluid communication with the lumen of the magazine tube when the gate is in the open configuration.

In an aspect of the second embodiment, and in combination with any other aspects herein, the disclosure provides that the distal end of the cannula is beveled.

In an aspect of the second embodiment, and in combination with any other aspects herein, the disclosure provides that the push rod is attached to a shuttle body and the shuttle body is coupled to a spring, the spring including a non-extended configuration and an extended configuration and being biased to the non-extended configuration. In an aspect of the second embodiment, and in combination with any other aspects herein, the disclosure provides that the spring is coiled in the non-extended configuration. In an aspect of the second embodiment, and in combination with any other aspects herein, the disclosure provides that the actuator in a non-deployed position holds the shuttle body such that the spring is in the extended configuration. In an aspect of the second embodiment, and in combination with any other aspects herein, the disclosure provides that actuation of the actuator from the non-deployed position to a deployed position releases the shuttle body and permits the spring to resume the non-extended configuration. In an aspect of the second embodiment, and in combination with any other aspects herein, the disclosure provides when the actuator is in the non-deployed position, the actuator contacts and engages the shuttle body and when the actuator is in the deployed position, the actuator does not contact the shuttle body and is disengaged therefrom.

In an aspect of the second embodiment, and in combination with any other aspects herein, the disclosure provides that the cannula and the magazine tube are coaxially aligned and a transition gap extends between the outlet of the magazine tube and an inlet of the cannula. In an aspect of the second embodiment, and in combination with any other aspects herein, the disclosure provides that a portion of the gate is disposed within the transition gap when the gate is in the closed configuration and the portion of the gate is not disposed within the transition gap when the gate is in the open configuration.

In an aspect of the second embodiment, and in combination with any other aspects herein, the disclosure provides that the push rod is attached to a shuttle body. A drag wire is attached to the shuttle body. The injector further includes a shuttle decelerator that slows down translation of the shuttle body and push rod attached thereto. In an aspect of the second embodiment, and in combination with any other aspects herein, the disclosure provides that the shuttle decelerator includes a plurality of bosses that form a sinusoidal path and interaction between the drag wire and the plurality bosses creates friction. In an aspect of the second embodiment, and in combination with any other aspects herein, the disclosure provides that the drag wire is formed from stainless steel and the plurality of bosses are formed from a plastic material.

According to a third embodiment hereof, the present disclosure provides an injector including a housing, a push rod disposed at least partially within the housing, a magazine tube disposed within the housing, a cannula having a distal end that is disposed outside of the housing and is configured to be inserted into an eye, and an actuator. The magazine tube has an inlet, an outlet, and a lumen extending from the inlet to the outlet. The magazine tube is configured to slidingly receive at least one implant therein, and the push rod is configured to be slidingly received within the lumen of the magazine tube. Actuation of the actuator causes translation of the pushrod through the magazine tube and the cannula. The magazine tube is configured to hold at least three implants and the injector is configured to deliver the at least three implants via a single actuation of the actuator. A speed of delivery of the at least three implants is controlled such that the speed of delivery is between 2 and 12 seconds.

In an aspect of the third embodiment, and in combination with any other aspects herein, the disclosure provides that the injector further includes a gate disposed within the housing. The gate has a closed configuration in which it covers the outlet of the magazine tube and an open configuration in which it does not cover the outlet of the magazine tube. A lumen of the cannula is in fluid communication with the lumen of the magazine tube when the gate is in the open configuration. Actuation of the actuator moves the gate from the closed configuration to the open configuration. In an aspect of the third embodiment, and in combination with any other aspects herein, the disclosure provides that the cannula and the magazine tube are coaxially aligned and a transition gap extends between the outlet of the magazine tube and an inlet of the cannula. In an aspect of the third embodiment, and in combination with any other aspects herein, the disclosure provides that a portion of the gate is disposed within the transition gap when the gate is in the closed configuration and the portion of the gate is not disposed within the transition gap when the gate is in the open configuration.

In an aspect of the third embodiment, and in combination with any other aspects herein, the disclosure provides that the injector further includes a safety cap that is configured to be removably coupled to the housing to cover the distal end of the cannula when the safety cap is coupled to the housing. The safety cap includes a tab that extends into a slot formed in the actuator when the safety cap is coupled to the housing to prevent actuation of the actuator.

In an aspect of the third embodiment, and in combination with any other aspects herein, the disclosure provides the housing has a generally tubular construction with an asymmetrical fin that includes a height that is greater than a height of the remaining length of the housing.

In an aspect of the third embodiment, and in combination with any other aspects herein, the disclosure provides that the distal end of the cannula is beveled.

In an aspect of the third embodiment, and in combination with any other aspects herein, the disclosure provides the push rod is attached to a shuttle body that is slidingly disposed within the housing and the shuttle body is coupled to a spring, the spring including a non-extended configuration and an extended configuration and being biased to the non-extended configuration. In an aspect of the third embodiment, and in combination with any other aspects herein, the disclosure provides that the spring is coiled in the non-extended configuration. In an aspect of the third embodiment, and in combination with any other aspects herein, the disclosure provides that the actuator in a non-deployed position holds the shuttle body such that the spring is in the extended configuration. In an aspect of the third embodiment, and in combination with any other aspects herein, the disclosure provides actuation of the actuator from the non-deployed position to a deployed position releases the shuttle body and permits the spring to resume the non-extended configuration. In an aspect of the third embodiment, and in combination with any other aspects herein, the disclosure provides when the actuator is in the non-deployed position, the actuator contacts and engages the shuttle body and when the actuator is in the deployed position, the actuator does not contact the shuttle body and is disengaged therefrom. In an aspect of the third embodiment, and in combination with any other aspects herein, the disclosure provides that the actuator is configured to rotate relative to the shuttle body to transition between the non-deployed position and the deployed position.

In an aspect of the third embodiment, and in combination with any other aspects herein, the disclosure provides that the housing includes a window formed thereon to permit visual feedback relating to the translation of the pushrod. In an aspect of the third embodiment, and in combination with any other aspects herein, the disclosure provides that the push rod is attached to a shuttle body that is slidingly disposed within the housing and an outer surface of the shuttle body includes a status indicator thereon for providing the visual feedback through the window.

In an aspect of the third embodiment, and in combination with any other aspects herein, the disclosure provides that the push rod is attached to a shuttle body that is slidingly disposed within the housing. A drag wire is attached to the shuttle body to control the speed of delivery of the at least three implants. The injector further includes a shuttle decelerator disposed within the housing, and the shuttle decelerator is configured to receive the drag wire within a sinusoidal path thereof. In an aspect of the third embodiment, and in combination with any other aspects herein, the disclosure provides the sinusoidal path of the shuttle decelerator is defined by a plurality of bosses and interaction between the drag wire and the plurality of bosses creates friction that slows down translation of the shuttle body and push rod attached thereto. In an aspect of the third embodiment, and in combination with any other aspects herein, the disclosure provides the drag wire is formed from stainless steel and the plurality of bosses are formed from a plastic material.

In an aspect of the third embodiment, and in combination with any other aspects herein, the disclosure provides that the push rod is formed from stainless steel.

In an aspect of the third embodiment, and in combination with any other aspects herein, the disclosure provides that the speed of delivery of the at least three implants is controlled such that the speed of delivery is between 3 and 10 seconds.

In an aspect of the third embodiment, and in combination with any other aspects herein, the disclosure provides that the speed of delivery of the at least three implants is controlled such that the speed of delivery is between 4 and 9 seconds.

In an aspect of the third embodiment, and in combination with any other aspects herein, the disclosure provides that the injector further includes a rotary damper disposed within the housing, the rotary damper being coupled to the push rod and configured to slow down the rate at which the push rod moves within the housing.

In an aspect of the third embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.035 in-lbs, and the injector has an injection speed of between 4 and 9 seconds.

In an aspect of the third embodiment, and in combination with any other aspects herein, the disclosure provides that the push rod is attached to a shuttle body that is slidingly disposed within the housing and the shuttle body is coupled to a spring, and wherein the spring has a spring constant for load of 0.4 pounds of force (0.4 lbf), the rotary damper has a damping torque of 0.026 in-lbs, and the injector has an injection speed of between 2.5 and 7.5 seconds.

In an aspect of the third embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.026 in-lbs, and the injector has an injection speed of between 1.5 and 6.5 seconds.

According to a fourth embodiment hereof, the present disclosure provides an injector including a housing including a window formed thereon, a push rod disposed at least partially within the housing, a magazine tube disposed within the housing, a cannula having a distal end that is disposed outside of the housing and is configured to be inserted into an eye, and an actuator. The magazine tube has an inlet, an outlet, and a lumen extending from the inlet to the outlet. The magazine tube is configured to slidingly receive at least one implant therein, and the push rod is configured to be slidingly received within the lumen of the magazine tube. Actuation of the actuator causes translation of the pushrod through the magazine tube and the cannula. The window permits visual feedback of translation of the pushrod through the housing, the visual feedback providing an indication that delivery of the at least one implant is complete.

In an aspect of the fourth embodiment, and in combination with any other aspects herein, the disclosure provides that the injector further includes a gate disposed within the housing. The gate has a closed configuration in which it covers the outlet of the magazine tube and an open configuration in which it does not cover the outlet of the magazine tube. A lumen of the cannula is in fluid communication with the lumen of the magazine tube when the gate is in the open configuration. Actuation of the actuator moves the gate from the closed configuration to the open configuration. In an aspect of the fourth embodiment, and in combination with any other aspects herein, the disclosure provides that the cannula and the magazine tube are coaxially aligned and a transition gap extends between the outlet of the magazine tube and an inlet of the cannula. In an aspect of the fourth embodiment, and in combination with any other aspects herein, the disclosure provides that a portion of the gate is disposed within the transition gap when the gate is in the closed configuration and the portion of the gate is not disposed within the transition gap when the gate is in the open configuration.

In an aspect of the fourth embodiment, and in combination with any other aspects herein, the disclosure provides that the injector further includes a safety cap that is configured to be removably coupled to the housing to cover the distal end of the cannula when the safety cap is coupled to the housing. The safety cap includes a tab that extends into a slot formed in the actuator when the safety cap is coupled to the housing to prevent actuation of the actuator.

In an aspect of the fourth embodiment, and in combination with any other aspects herein, the disclosure provides the housing has a generally tubular construction with an asymmetrical fin that includes a height that is greater than a height of the remaining length of the housing.

In an aspect of the fourth embodiment, and in combination with any other aspects herein, the disclosure provides that the distal end of the cannula is beveled.

In an aspect of the fourth embodiment, and in combination with any other aspects herein, the disclosure provides the push rod is attached to a shuttle body that is slidingly disposed within the housing and the shuttle body is coupled to a spring, the spring including a non-extended configuration and an extended configuration and being biased to the non-extended configuration. In an aspect of the fourth embodiment, and in combination with any other aspects herein, the disclosure provides that the spring is coiled in the non-extended configuration. In an aspect of the fourth embodiment, and in combination with any other aspects herein, the disclosure provides that the actuator in a non-deployed position holds the shuttle body such that the spring is in the extended configuration. In an aspect of the fourth embodiment, and in combination with any other aspects herein, the disclosure provides actuation of the actuator from the non-deployed position to a deployed position releases the shuttle body and permits the spring to resume the non-extended configuration. In an aspect of the fourth embodiment, and in combination with any other aspects herein, the disclosure provides when the actuator is in the non-deployed position, the actuator contacts and engages the shuttle body and when the actuator is in the deployed position, the actuator does not contact the shuttle body and is disengaged therefrom. In an aspect of the fourth embodiment, and in combination with any other aspects herein, the disclosure provides that the actuator is configured to rotate relative to the shuttle body to transition between the non-deployed position and the deployed position.

In an aspect of the fourth embodiment, and in combination with any other aspects herein, the disclosure provides that the push rod is attached to a shuttle body that is slidingly disposed within the housing. A drag wire is attached to the shuttle body. The injector further includes a shuttle decelerator disposed within the housing, and the shuttle decelerator is configured to receive the drag wire within a sinusoidal path thereof. In an aspect of the fourth embodiment, and in combination with any other aspects herein, the disclosure provides the sinusoidal path of the shuttle decelerator is defined by a plurality of bosses and interaction between the drag wire and the plurality of bosses creates friction that slows down translation of the shuttle body and push rod attached thereto. In an aspect of the fourth embodiment, and in combination with any other aspects herein, the disclosure provides the drag wire is formed from stainless steel and the plurality of bosses are formed from a plastic material.

In an aspect of the fourth embodiment, and in combination with any other aspects herein, the disclosure provides that the push rod is formed from stainless steel.

In an aspect of the fourth embodiment, and in combination with any other aspects herein, the disclosure provides that the push rod is attached to a shuttle body that is slidingly disposed within the housing and an outer surface of the shuttle body includes at least one status indicator thereon for providing the visual feedback through the window. In an aspect of the fourth embodiment, and in combination with any other aspects herein, the disclosure provides that the at least one status indicator includes a first status indicator and a second status indicator. The first status indicator is disposed proximal to the second status indicator. The first status indicator is displayed through the window prior to actuation of the actuator and the second status indicator is displayed through the window when the delivery of the at least one implant is complete. In an aspect of the fourth embodiment, and in combination with any other aspects herein, the disclosure provides that the at least one status indicator further includes a third status indicator disposed between the first status indicator and the second status indicator, and the second status indicator is disposed through the window while the shuttle body is moving within the housing. In an aspect of the fourth embodiment, and in combination with any other aspects herein, the disclosure provides that the first status indicator is a first color, the second status indicator is a second color, and the third status indicator is a third color.

In an aspect of the fourth embodiment, and in combination with any other aspects herein, the disclosure provides that the injector further includes a rotary damper disposed within the housing, the rotary damper being coupled to the push rod and configured to slow down the rate at which the push rod moves within the housing.

In an aspect of the fourth embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.035 in-lbs, and the injector has an injection speed of between 4 and 9 seconds.

In an aspect of the fourth embodiment, and in combination with any other aspects herein, the disclosure provides that the push rod is attached to a shuttle body that is slidingly disposed within the housing and the shuttle body is coupled to a spring, and wherein the spring has a spring constant for load of 0.4 pounds of force (0.4 lbf), the rotary damper has a damping torque of 0.026 in-lbs, and the injector has an injection speed of between 2.5 and 7.5 seconds.

In an aspect of the fourth embodiment, and in combination with any other aspects herein, the disclosure provides that the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.026 in-lbs, and the injector has an injection speed of between 1.5 and 6.5 seconds.

According to a fifth embodiment hereof, the present disclosure provides a method of using an injector to deliver at least one implant into an eye. A distal tip of the injector is positioned adjacent to an injection site of the eye. The injector includes a push rod, a magazine tube having an inlet, an outlet, and a lumen extending from the inlet to the outlet, the magazine tube having the at least one implant disposed therein, a gate in a closed configuration in which it covers the outlet of the magazine tube, a cannula, an actuator, and a status indicator. The distal end of the injector is advanced into tissue of the eye at the injection site. The actuator is actuated to deliver the at least one implant into the tissue of the eye. Actuation of the actuator moves the gate from the closed configuration to an open configuration in which the gate does not cover the outlet of the magazine tube and actuation of the actuator also causes translation of the pushrod through the magazine tube and the cannula to push the at least one implant. The position of the distal end of the injector is maintained within the tissue of the eye until the status indicator of the injector indicates completion of implant deliver. The injector is removed from the tissue of the eye after the status indicator of the injector indicates completion of implant delivery.

In an aspect of the fifth embodiment, and in combination with any other aspects herein, the disclosure provides that the at least one implant includes exactly three implants.

In an aspect of the fifth embodiment, and in combination with any other aspects herein, the disclosure provides that the at least one implant is used to treat chronic non-infectious uveitis affecting the posterior segment of the eye in an eye in need thereof.

In an aspect of the fifth embodiment, and in combination with any other aspects herein, the disclosure provides that the tissue of the eye includes a vitreous of the eye.

In an aspect of the fifth embodiment, and in combination with any other aspects herein, the disclosure provides that the implant is an intravitreal implant comprising about 0.18 mg fluocinolone acetonide.

In an aspect of the fifth embodiment, and in combination with any other aspects herein, the disclosure provides that the implant also comprises polyvinyl alcohol, silicone adhesive, a polyimide tube and may comprise water.

In an aspect of the fifth embodiment, and in combination with any other aspects herein, the disclosure provides that the at least one implant is used to treat a retinal disease in an eye in need thereof.

In an aspect of the fifth embodiment, and in combination with any other aspects herein, the disclosure provides that the retinal disease is selected from wet AMD, diabetic retinopathy, diabetic macular edema and retinal vein occlusion.

In an aspect of the fifth embodiment, and in combination with any other aspects herein, the disclosure provides that the implant is an intravitreal implant comprises about 400 µg to about 2800 µg vorolanib.

In an aspect of the fifth embodiment, and in combination with any other aspects herein, the disclosure provides that the implant also comprises polyvinyl alcohol.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an exploded perspective view of an injector according to an embodiment hereof, wherein the injector is in a non-deployed state and a safety cap is not coupled to the injector.

FIG. 2 is an enlarged perspective view of a distal end portion of the injector of FIG. 1, wherein the injector is in the non-deployed state and the safety cap is not coupled to the injector.

FIG. 3 is a side view of the injector of FIG. 1, wherein the injector is in the non-deployed state and the safety cap is coupled to the injector.

FIG. 4 is an enlarged perspective view of the distal end portion of the injector of FIG. 1, wherein the injector is in the non-deployed state and the safety cap is coupled to the injector.

FIG. 5 is a perspective view of the injector of FIG. 1, wherein the injector is in the non-deployed state and a housing of the injector is removed for sake of illustration.

FIG. 5A is a sectional view of FIG. 5, taken along line A-A of the FIG. 5.

FIG. 6 is a perspective view of the injector of FIG. 1, wherein the injector is in a deployed state.

FIG. 7 is a perspective view of the injector of FIG. 1, wherein the injector is in the deployed state and the housing of the injector is removed for sake of illustration.

FIG. 7A is a sectional view of FIG. 7, taken along line A-A of the FIG. 7.

FIG. 8A is a perspective view of a window chassis of the injector of FIG. 1, wherein the window chassis is shown removed from the injector for sake of illustration.

FIG. 8B is a side view of the window chassis of FIG. 8A.

FIG. 9 is a perspective view of a magazine subassembly of the injector of FIG. 1, the magazine subassembly including a magazine tube, a magazine tube mount, a gate, a cannula, and a cannula mount, wherein the magazine subassembly is shown removed from the injector for sake of illustration.

FIG. 10 is a perspective view of the cannula and the cannula mount of FIG. 9.

FIG. 11 is another perspective view of the cannula and the cannula mount of FIG. 9, wherein the cannula mount is shown in phantom.

FIG. 12 is a perspective view of the magazine tube, the magazine tube mount, and the gate of FIG. 9.

FIG. 13 is an end view of FIG. 12.

FIG. 14 is the end view of FIG. 13 with the gate removed for sake of illustration.

FIG. 15 is a perspective view of the magazine tube of FIG. 12, wherein magazine tube is shown in phantom and is shown holding three implants.

FIG. 16A is an enlarged sectional view of the magazine subassembly of FIG. 9 when the gate is in a closed configuration.

FIG. 16B is an enlarged sectional view of the magazine subassembly of FIG. 9 when the gate is in an open configuration.

FIG. 17 is a perspective view of the gate of the magazine subassembly of FIG. 9.

FIG. 18 is a perspective view of a shuttle subassembly of the injector of FIG. 1, the shuttle subassembly including a shuttle body, a pushrod, a spring, and a drag wire, wherein the shuttle subassembly is shown removed from the injector for sake of illustration.

FIG. 18A is a sectional view of FIG. 18, taken along line A-A of FIG. 18.

FIG. 19 is a perspective view of the spring of the shuttle subassembly of FIG. 18.

FIG. 20 is an enlarged perspective view of the actuator of the injector of FIG. 1 when the injector is in the non-deployed state.

FIG. 21 is an enlarged side view of the actuator of the injector of FIG. 1 when the injector is in the non-deployed state.

FIG. 22 is an enlarged side view of the actuator of the injector of FIG. 1 when the injector is in the deployed state.

FIG. 23 is a perspective view of the window chassis of FIG. 8A attached to an actuator chassis of the injector, wherein the window chassis and the actuator chassis are shown removed from the injector for sake of illustration.

FIG. 24 is a perspective view of the magazine assembly of FIG. 9 coupled to the window chassis and the actuator chassis of FIG. 23, wherein the magazine assembly, the window chassis and the actuator chassis are shown removed from the injector for sake of illustration.

FIG. 25 is a perspective view of the push rod coupled to the window chassis and the actuator chassis of FIG. 23, wherein the push rod, the window chassis and the actuator chassis are shown removed from the injector for sake of illustration.

FIG. 26A is a perspective view of the actuator chassis of FIG. 23 and the actuator of the injector, wherein the actuator chassis and the actuator are shown removed from the injector for sake of illustration and the actuator is in the non-deployed state.

FIG. 26B is a perspective view of the actuator chassis of FIG. 23 and the actuator of the injector, wherein the actuator chassis and the actuator are shown removed from the injector for sake of illustration and the actuator is in the deployed state.

FIG. 27 is a perspective view of the actuator chassis of FIG. 23.

FIG. 28 is a perspective view of the actuator chassis of FIG. 23 and the actuator disposed therein when the injector is in the non-deployed state.

FIG. 29 is a perspective view of the actuator chassis of FIG. 23 and the actuator disposed therein when the injector is in the deployed state.

FIG. 30A is a perspective view of the actuator and the magazine subassembly of the injector of FIG. 1 to illustrate the relative positioning of the actuator and the gate when the injector is in the non-deployed state.

FIG. 30B is an enlarged sectional view to illustrate the relative positioning of the actuator and the gate when the injector is in the non-deployed state.

FIG. 31 is an enlarged sectional view to illustrate the relative positioning of the actuator and the gate when the injector is in the deployed state.

FIG. 32 is an enlarged sectional view to illustrate the relative positioning of the actuator and the shuttle subassembly when the injector is in the non-deployed state.

FIG. 33 is an enlarged sectional view to illustrate the relative positioning of the actuator and the shuttle subassembly when the injector is in the deployed state.

FIG. 34 is an enlarged perspective view of the injector of FIG. 1, the housing and the window chassis removed from the injector for sake of illustration, wherein the injector is shown in the deployed state and the shuttle subassembly is shown in phantom.

FIG. 35 is an enlarged sectional view of the shuttle subassembly of the injector of FIG. 1, wherein the injector further includes a shuttle decelerator.

FIG. 36 is a perspective view of the shuttle decelerator of FIG. 35, wherein the shuttle decelerator is shown removed from the injector for sake of illustration.

FIG. 37 is an enlarged perspective view of the drag wire of the shuttle subassembly disposed through the shuttle decelerator.

FIG. 38 is a perspective view of an injector according to another embodiment hereof, wherein the injector includes a rotary damper.

FIG. 39 is a perspective view of a shuttle subassembly of the injector of FIG. 38, wherein the shuttle subassembly is removed from the injector for sake of illustration.

FIG. 40 is a method of using an injector according to an embodiment hereof.

### DETAILED DESCRIPTION OF THE INVENTION

Specific embodiments of the present invention are now described with reference to the figures, wherein like reference numbers indicate identical or functionally similar elements. The following detailed description is merely exemplary in nature and is not intended to limit the invention or the application and uses of the invention. Although the description of the invention is primarily in the context of delivering implants into eye tissue, the invention may also be used to delivery other implants where it is deemed useful. The term “injector” is broadly intended to comprise all types of dispensing apparatus and the injector of the present disclosure is not restricted to medical use. In addition, the term “injector” may be used interchangeably with the term “applicator” herein and the term “injecting” may be used interchangeably with the term “inserting” herein. Furthermore, there is no intention to be bound by any expressed or implied theory presented in the preceding technical field, background, brief summary or the following detailed description.

In the present disclosure, the term “proximal” is used to refer to that portion of an element closest to the physician using the device to inject an implant into an injection site. The term “distal” is used herein to refer to that portion of an element farthest from the physician’s hand, and closest to the injection site, when the injector is utilized to inject an implant. In addition, as used herein, the terms “a” or “an” is used to refer to one or more. For example, “an implant” is used herein to refer to one or more implants. In addition, the term “implant” may be used interchangeably with the term “insert” herein.

In an embodiment, the injector of the present disclosure is configured for intraocular drug delivery and is used to deliver one or more implants or payloads into the eye. In an embodiment, the injector is configured to deliver multiple implants into the posterior segment of a human eye. The implant may include a therapeutically effective amount of one or more drugs, and may be of any solid composition, e.g., for releasing drug or other agents. Such devices typically can be implanted into any number of locations in tissue and can be designed such that a controlled amount of desired drug or therapeutic can be released over time. In certain embodiments, the implant includes a therapeutic agent and a polymer. The therapeutic agent may comprise a steroid or a biologic. For example, the therapeutic agent may comprise bevacizumab or ranibizumab. In preferred embodiments, the therapeutic agent comprises a corticosteroid, such as fluocinolone acetonide. In some embodiments, the longitudinal length of the implant is between 0.1 and 0.6 centimeters. The implant can be delivered through a cannula of the injector corresponding to 21-gauge cannula or smaller, and therefore has a cross-sectional diameter of 0.66 mm or less. The injector may be used to position the implant at a desired implantation site, e.g., in the vitreous cavity of the eye. For such embodiments, as will be described in more detail herein, the injector may be positioned near the eye and the cannula of the injector may be positioned through the sclera and into the vitreous of the eye for placement of the implant. Once the implant is delivered into the eye, the cannula can be withdrawn. Administering the implant may comprise injecting the implant into an eye of a subject, such as inserting the implant into the posterior segment of the eye, anterior or posterior chambers of the eye, or other areas of the eye including intraretinal, intravitreal, subretinal, intrachoroidal, suprachoroidal, intrascleral, suprascleral, episcieral, subconjunctival, intracorneal or epicorneal spaces. For example, the implant may be injected into the aqueous humor or, preferably, into the vitreous humor of an eye (injecting intravitreally).

In an aspect of the first embodiment, and in combination with any other aspects herein, the disclosure provides a method of preventing or treating an ocular condition or disease of the eye in an eye in need thereof comprising using the injector of the first embodiment to administer an implant containing an active pharmaceutical ingredient (API). In some embodiments, the implant is administered to treat an anterior ocular condition. In other embodiments, it may be administered to treat a posterior ocular condition. In some embodiments, the implant is administered to prevent an anterior ocular condition. In other embodiments, it may be administered to prevent a posterior ocular condition.

An “anterior ocular condition” is a disease, ailment, or condition that affects or involves an anterior (i.e., front of the eye, also referred to as the anterior segment) ocular region or structure, such as a periocular muscle or an eye lid, or a fluid located anterior to the posterior wall of the lens capsule or ciliary muscles. Thus, an anterior ocular condition can affect or involve the conjunctiva, the cornea, the anterior chamber, the iris, the posterior chamber (located between the iris and lens), the lens or the lens capsule and blood vessels and nerve which vascularize or innervate an anterior ocular region or site.

An anterior ocular condition can include a disease, ailment or condition such as, but not limited to, glaucoma.

A “posterior ocular condition” is a disease, ailment or condition that primarily affects or involves a posterior (i.e., back of the eye, also referred to as the posterior segment) ocular region or structure, such as choroid or sclera (in a position posterior to a plane through the posterior wall of the lens capsule), vitreous, vitreous chamber, retina, optic nerve or optic disc, and blood vessels and nerves that vascularize or innervate a posterior ocular region or site.

A posterior ocular condition can include a disease, ailment or condition such as, but not limited to, acute macular neuroretinopathy; Behcet’s disease; geographic atrophy; choroidal neovascularization; diabetic uveitis; histoplasmosis; infections, such as fungal, bacterial, or viral-caused infections; macular degeneration, such as neovascular macular degeneration, acute macular degeneration, non-exudative age related macular degeneration and exudative age related macular degeneration; edema, such as macular edema, cystoids macular edema and diabetic macular edema; multifocal choroiditis; ocular trauma which affects a posterior ocular site or location; ocular tumors; retinal disorders, such as retinal vein occlusion, central retinal vein occlusion, diabetic retinopathy (including proliferative diabetic retinopathy), proliferative vitreoretinopathy (PVR), hypertensive retinopathy, retinal arterial occlusive disease such as central retinal artery occlusion (CRAO) and branch retinal artery occlusion (BRAO), retinal detachment, uveitic retinal disease; sympathetic ophthalmia; Vogt Koyanagi-Harada (VKH) syndrome; uveal diffusion; a posterior ocular condition caused by or influenced by an ocular laser treatment; or posterior ocular conditions caused by or influenced by a photodynamic therapy, photocoagulation, radiation retinotherapy, epiretinal membrane disorders, branch retinal vein occlusion, anterior ischemic optic neuropathy, non-retinopathy diabetic retinal dysfunction, and retinitis pigmentosa. Glaucoma may also be considered a posterior ocular condition because the therapeutic goal is to prevent the loss of or reduce the occurrence of loss of vision due to damage to or loss of retinal cells or optic nerve cells (e.g., via neuroprotection).

In certain embodiments, the implants are administered to prevent or treat macular degeneration in an eye in need thereof, e.g., age-related macular degeneration (“AMD”), such as dry AMD and wet AMD. The implant may be administered to prevent the death of retinal pigment epithelial cells. The implant may be administered to inhibit angiogenesis. In some embodiments, the implants are administered to prevent or treat vision loss in an eye, such as vision loss associated with macular degeneration. In addition, the implant may be administered to prevent or delay the progression of dry AMD to wet AMD. In some embodiments, the implant is administered to prevent or treat retinal vein occlusion in an eye in need thereof, e.g., central retinal vein occlusion (“CRVO”) or branch retinal vein occlusion (“BRVO”). In other embodiments, the implants may be administered to prevent or treat non-ischemic retinal vein occlusion or ischemic retinal vein occlusion. In yet other embodiments, the implant is administered to treat diabetic retinopathy in an eye in need thereof.

The API in the implant to be administered for a particular ocular condition or disease is selected based on the suitability of the API for that ocular condition.

The implant of the present invention may be used to deliver various classes of APIs. Examples of these classes of APIs and of specific APIs include the following:

In some embodiments, the API is a vascular endothelial growth factor (VEGF) inhibitor (also sometimes referred to as an anti-VEGF), a kinase inhibitor such as a tyrosine kinase (TKI) inhibitor, a vascular endothelial protein tyrosine phosphatase (VE-PTP) inhibitor, an Ang-1 inhibitor, an Ang-2 inhibitor, a Tie-2 activator, a Tie-2 agonist, or an mTOR inhibitor. API’s having one or more of these activities include altiratinib, rebastinib, afatinib, alectinib, apatinib, ASP-3026, axitinib, bafetinib, baricitinib, binimetinib, bosutinib, brigatinib, cabozantinib, canertinib, cediranib, CEP-11981, CEP-37440, ceritinib, cobimetinib, copanlisib, crenolanib, crizotinib, CYT387, dabrafenib, damnacanthal, dasatinib, doramapimod, enterctinib, erlotinib, everolimus, filgotinib, foretinib, fostamatinib, gefitinib, grandinin, ibrutinib, icotinib, idelalisib, imatinib, IPI-145, JSI-124, lapatinib, lenvatinib, lestaurtinib, linifanib, masitinib, motesanib, mubritinib, neratinib, nilotinib, nintedanib, pacritinib, palbociclib, pazopanib, pegaptanib, perifosine, pexmetinib, PF-06463922, ponatinib, PX-866, quizartinib, radotinib, razuprotafib (AKB-9778), regorafenib, ruxolitinib, selumetinib, semaxanib, sirolimus, sorafenib, sorafenib tosylate, staurosporine, sunitinib, sunitinib malate, SU6656, temsirolimus, TG101348, tivozanib, toceranib, tofacitinib, trametinib, TSR-011, vandetanib, vatalanib, vemurafenib, vorolanib, and X-396.

In some embodiments the API may be a steroidal anti-inflammatory agent such as a steroid or corticosteroid, non-limiting examples of which are fluocinolone acetonide, hydrocortisone, hydrocortisone acetate, triamcinolone acetonide, methylprednisolone, dexamethasone, medrysone, methylprednisolone, prednisolone 21-phosphate, prednisolone acetate, fluoromethalone and betamethasone.

In other embodiments, the API is a prostaglandin or a prostaglandin analog or agonist, such as bimatoprost, latanoprost, latanoprostene bunod, tafluprost, or travoprost.

In yet other embodiments, the API is an alpha-2 adrenergic receptor agonist, such as brimonidine, brimonidine tartrate, or brimonidine pamoate.

In some aspects, the API is a beta-blocker such as timolol.

In other aspects, the API is a carbonic anhydrase inhibitor (CAI) such as acetazolamide, brinzolamide, dorzolamide, or methazolamide.

In other aspects, the API is a rho khinase inhibitor such as netarsudil.

Non-steroidal anti-inflammatory drugs (NSAIDs) are also contemplated. NSAIDS include diclofenac, etoldolac, fenoprofen, floctafenine, flurbiprofen, ibuprofen, indoprofen, ketoprofen, ketorolac, lomoxicam, morazone, naproxen, perisoxal, pirprofen, pranoprofen, suprofen, suxibuzone, tropesin, ximoprofen, zaltoprofen, zileuton, and zomepirac. COX-2 inhibitors such as valdecoxib, rofecoxib, and celecoxib are also contemplated.

In some embodiments, the API is a neuroprotectant such as nimodipine; an antibiotic such as tetracycline, chlortetracycline, bacitracin, neomycin, polymyxin, gramicidin, oxytetracycline, chloramphenicol, gentamycin, or erythromycin; or an antibacterial such as a sulfonamide, sulfacetamide, sulfamethizole, sulfisoxazole nitrofurazone or sodium propionate.

In another embodiment, the API is a compliment inhibitor, such as a C3 inhibitor, e.g., APL-2 (pegcetacoplan), or a C5 inhibitor.

Anesthetics and analgesic agents such as lidocaine and related compounds are also contemplated.

In some embodiments, the implant comprises more than one API.

In addition, the invention contemplates the use of analogs, derivatives, pharmaceutically acceptable salts, esters, prodrugs, codrugs, and protected forms thereof of the API.

The term “pharmaceutically acceptable salt” of a given compound refers to salts that retain the biological effectiveness and properties of the given compound, and which are not biologically or otherwise undesirable.

In some embodiments of the methods, the implant comprises a VEGF inhibitor, a kinase inhibitor such as a TKI inhibitor, a VE-PTP inhibitor, an Ang-1 inhibitor, an Ang-2 inhibitor, and/or a Tie-2 activator. In some embodiments the implant comprises vorolanib, or a pharmaceutically acceptable salt thereof. In other embodiments, the implant comprises axitinib, or a pharmaceutically acceptable salt thereof. In yet other embodiments, the implant comprises razuprotafib or a pharmaceutically acceptable salt or zwitterion thereof.

In other embodiments, the implant is administered to activate Tie-2. In some embodiments of this method, the implant comprises a Tie-2 activator. In a further embodiment, the Tie-2 activator is razuprotafib or a pharmaceutically acceptable salt or zwitterion thereof.

In some embodiments, the implant is administered to treat uveitis. In a further embodiment, the implant is administered to treat chronic non-infectious uveitis affecting the posterior segment of the eye. In some embodiments, the implant is administered to treat postoperative inflammation in the eye. In some embodiments of these methods, the implant comprises a steroidal anti-inflammatory agent. In an embodiment, the implant comprises a corticosteroid and is indicated for the treatment of chronic non-infectious uveitis affecting the posterior segment of the eye. The corticosteroid may be a synthetic corticosteroid such as but not limited to fluocinolone acetonide. The chemical name for fluocinolone acetonide is (6α, 11β, 16α)-6,9-difluoro-11,21-dihydroxy-16,17-[(1-methylethylidene)bis-(oxy)]-pregna-1,4-diene-3,20-dione. The implant may be a sterile non-bioerodible intravitreal implant containing 0.18 mg fluocinolone acetonide in a 36-month sustained-release drug delivery system. In an embodiment, the implant contains 0.18 mg of the active ingredient fluocinolone acetonide and the following inactive ingredients: polyimide tube, polyvinyl alcohol, silicone adhesive and water for injection. The implant may be configured to release fluocinolone acetonide at an initial rate of 0.25 mcg/day. In an embodiment, each implant may be approximately 3.5 mm (length) by 0.37 mm (width).

In some embodiments, the implant comprises a VEGF inhibitor. In some embodiments, the VEGF inhibitor is vorolanib or a pharmaceutically acceptable salt thereof.

In an embodiment, the implant is an intravitreal implant comprising about 100 µg to about 2800 µg vorolanib, about 400 µg to about 2800 µg, or about 400 µg to about 2000 µg. The implant may also include a polymer such as polyvinyl alcohol. In an embodiment, each implant has a length of about 3 mm to about 10 mm in length. In an embodiment, each implant has a length of about 6 mm to about 9 mm in length.

In an embodiment, the implant is configured to be implanted into other portions of the eye beyond the vitreous cavity of the eye. In an embodiment, the injector of the present disclosure is used to deliver one or more implants into the eye for the treatment of chronic non-infectious uveitis affecting the posterior segment of the eye. In some embodiments, the injector is used to deliver one or more implants into the eye for the treatment or prevention of a retinal disease. In some embodiments, the retinal disease is selected from wet AMD, diabetic retinopathy, diabetic macular edema and retinal vein occlusion.

The implant may be configured for treatment of patients with active or suspected ocular or periocular infections including most viral disease of the cornea and conjunctiva.

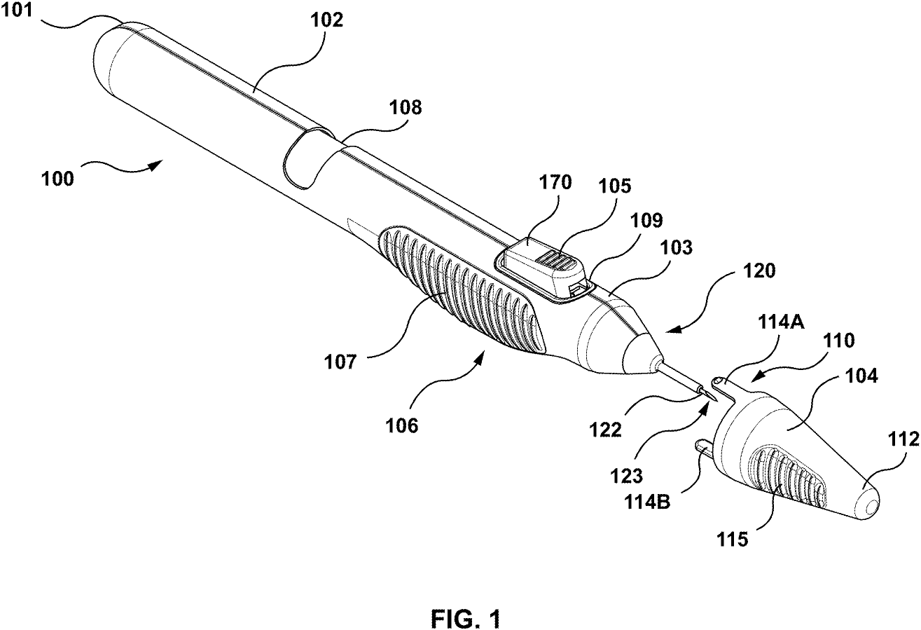

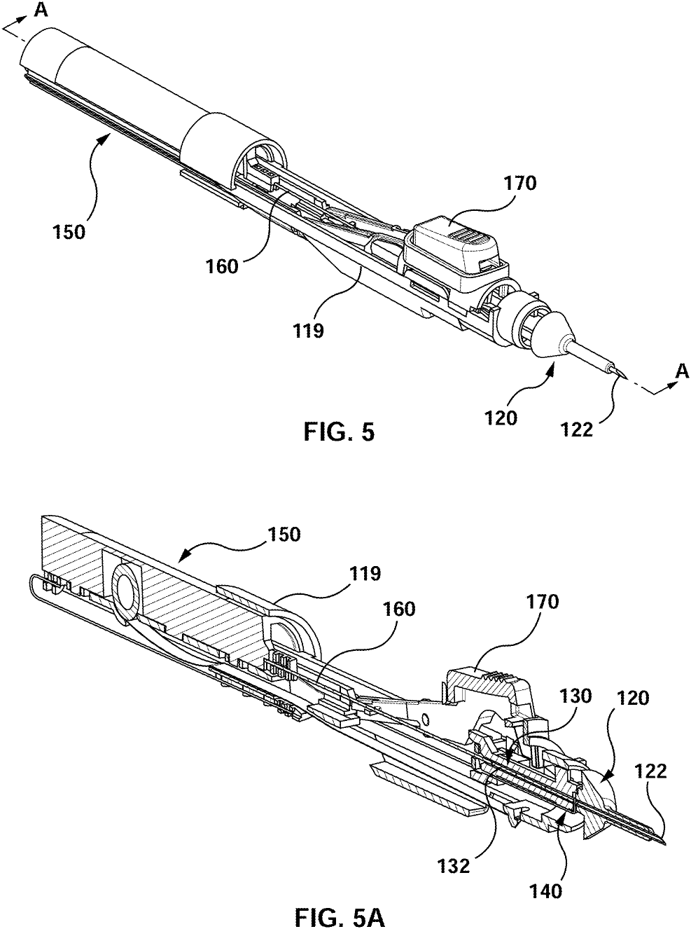

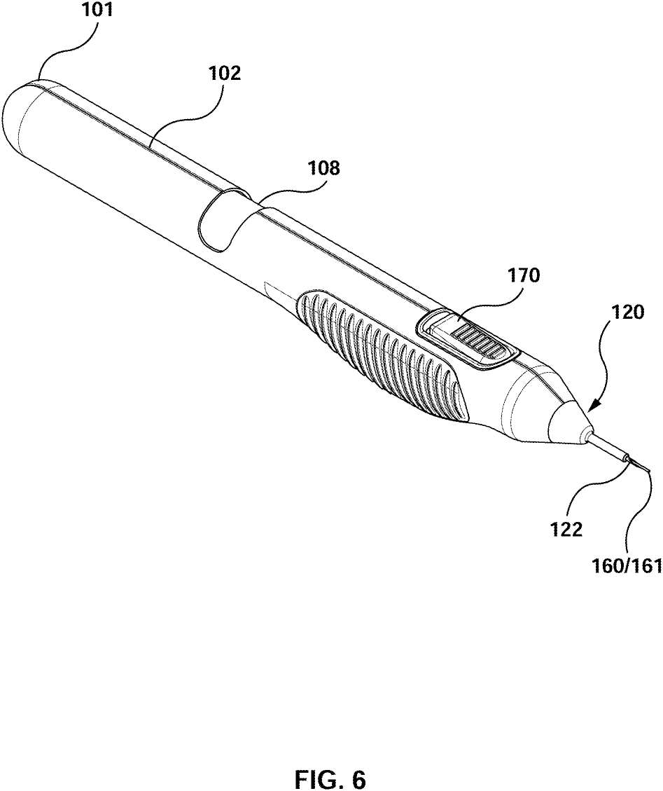

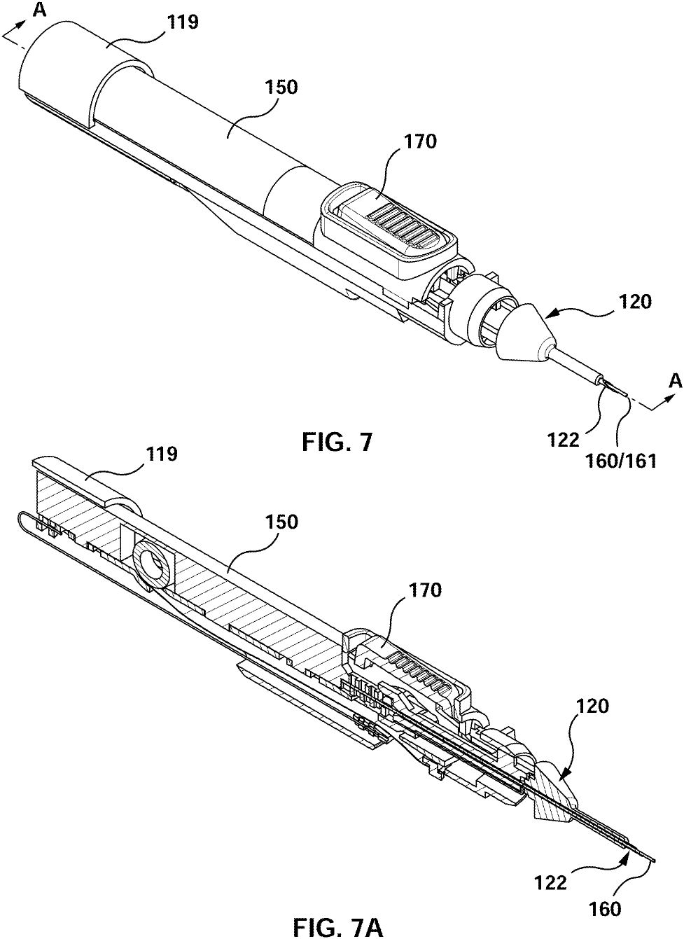

Turning now to the figures, an injector 100 according to the present disclosure will be described in more detail. FIGS. 1-5 illustrate the injector 100 in a non-deployed state, in which one or more implants are contained within the injector, while FIGS. 6-7A illustrate the injector 100 in a deployed state after the one or more implants are delivered into the eye. The injector 100 includes a housing 102 and a safety cap 104 removably coupled to the housing 102. FIG. 5 is a perspective view of the injector 100 in the non-deployed state with the housing 102 removed for sake of illustration to show the internal components thereof, and FIG. 5A is a sectional view taken along line A-A of the FIG. 5. Similarly, FIG. 7 is a perspective view of the injector 100 in the deployed state with the housing 102 removed for sake of illustration to show in the internal components thereof, and FIG. 7A is a sectional view taken along line A-A of the FIG. 7.

The injector 100 includes a magazine subassembly 130, a shuttle subassembly, and an actuator 170. The magazine subassembly 130 includes a cannula 122, a magazine tube 132, and a gate 140, while the shuttle subassembly 150 includes a push rod 160. The magazine tube 132, the gate 140, and the shuttle subassembly 150 are disposed within the housing 102. The cannula 122 is disposed partially within the housing 102 with a distal portion thereof distally extending from a distal end of the housing 102. More particularly, the cannula 122 has a distal end 123 which is not disposed within the housing 102 and is configured to be inserted into an eye. The distal end 123 of the cannula 122 defines the outlet of the cannula 122. The actuator 170 is also disposed partially within the housing 102, and is accessible to a user to operate the injector 100 from the non-deployed state of FIGS. 1-5 to the deployed state of FIGS. 6-7A.

As will be described in more detail herein, the magazine tube 132 of the magazine subassembly 130 is configured to slidingly receive or house at least one implant therein, and the push rod 160 is configured to be slidingly received within the magazine tube 132 and the cannula 122. The gate 140 of the magazine subassembly 130 has a closed configuration in which it covers or blocks an outlet of the magazine tube 132 and an open configuration in which it does not cover the outlet of the magazine tube 132 such that the outlet of the magazine tube 132 is exposed. The cannula 122 is in fluid communication with the magazine tube 132 when the gate 140 is in the open configuration. Actuation of the actuator 170 from a non-deployed position to a deployed position moves or displaces the gate 140 from the closed configuration to the open configuration. Actuation of the actuator 170 also results in or causes movement or translation of the pushrod 160 into and through the magazine tube 132 and the cannula 122 in order to deliver the at least one implant contained within the magazine tube 132 through the cannula 122 and into the eye.

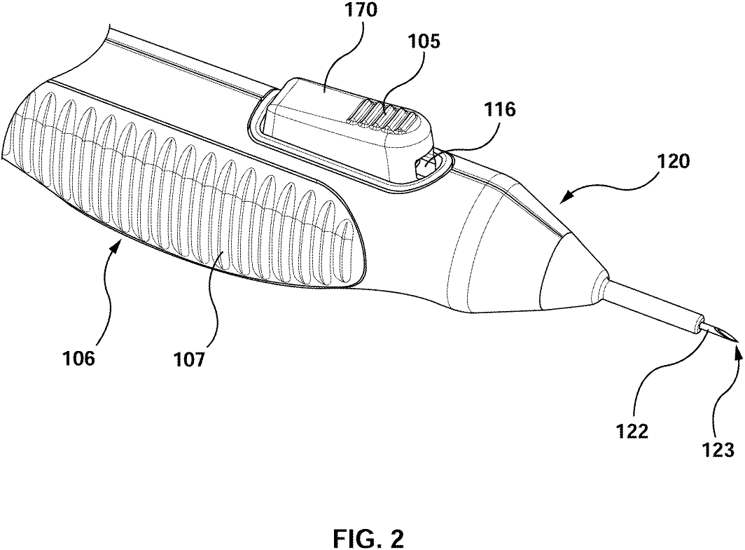

With reference to FIGS. 1-4, the housing 102 and the safety cap 104 of the injector 100 will now be described in more detail. FIGS. 1 and 2 illustrate the safety cap 104 not coupled to the housing 102 of the injector, with FIG. 2 being an enlarged perspective view of a distal end portion of the injector 100. The housing 102 includes a proximal end 101 and a distal end 103. The housing 102 is generally tubular or cylindrical in shape. In an embodiment, an outer diameter of the housing 102 ranges between 15 mm and 18 mm. The housing 102 is configured and sized such that the injector 100 may be operated with one hand in a typical clinical environment. The housing 102 may include an asymmetrical fin 106 near the distal end 103 thereof which is not cylindrical. The asymmetrical fin 106 has a relatively enlarged height or depth that is greater than the height or depth of the remaining length of the housing 102 which is cylindrical. For example, in an embodiment, the height or depth of the asymmetrical fin 106 is up to twice the height or depth of the remaining length of the housing 102. The width of the housing 102 is consistent along an entire length of the housing 102, regardless of whether or not the asymmetrical fin 106 is present. As such, in an embodiment, the width of the asymmetrical fin 106 is equal to the width of the remaining length of the housing 102 which is cylindrical. The asymmetrical fin 106 may extend between 20-40% of the entire length of the housing 102. The asymmetrical fin 106 includes a finger-gripping surface 107 thereon. Although FIGS. 1 and 2 show only one side of the injector 100, a similar finger-gripping surface is preferably included on the asymmetrical fin 106 on the opposing side of the housing 102. The shape and texture of the asymmetrical fin 106 are configured to permit a user to easily hold and grip the injector 100 to increase stability of the injector 100 during operation thereof. Each finger-gripping surface 107 may be formed from an elastomeric material and may include a plurality of ribs formed thereon. Any of a variety of shapes or configurations may be selected for the finger-gripping surface 107 to provide suitable finger placement during injector operation.

The housing 102 further includes a window 108 formed thereon. As will be described in more detail herein, the window 108 allows for visual feedback relating to the movement or translation of the shuttle subassembly 150 within the housing 102. Stated another way, a user can see movement of the shuttle subassembly 150 during operation of the injector 100. As will be explained in more detail herein, the shuttle subassembly 150 may include one or more status indicators on an outer surface thereof to alert the user of the relative positioning of the shuttle subassembly 150 within the housing 102. For example, the status indicators may include notations, symbols, instructions, colors, or the like. Prior to deployment or actuation of the actuator 170, for example, a first color or status indicator may be displayed to the user through the window 108 of the housing 102. The shuttle subassembly 150 moves relative to the housing 102 during operation of the injector 100, and thus a second color or status indicator may be displayed to the user through the window 108 of the housing 102 after deployment to provide visual feedback to the user that the deployment operation is complete.

The housing 102 also includes an opening 109 formed thereon. The actuator 170 extends through the opening 109 so as to be accessible to the user. When in a non-deployed position, as shown in FIGS. 1-4, the actuator 170 protrudes outwardly from the housing 102 for easy access for the user. The actuator 170 may include a finger-gripping surface 105 thereon. The finger-gripping surface 105 may be formed from an elastomeric material and may include a plurality of ribs formed thereon. Any of a variety of shapes or configurations may be selected for the finger-gripping surface 105 to provide suitable finger placement during injector operation.

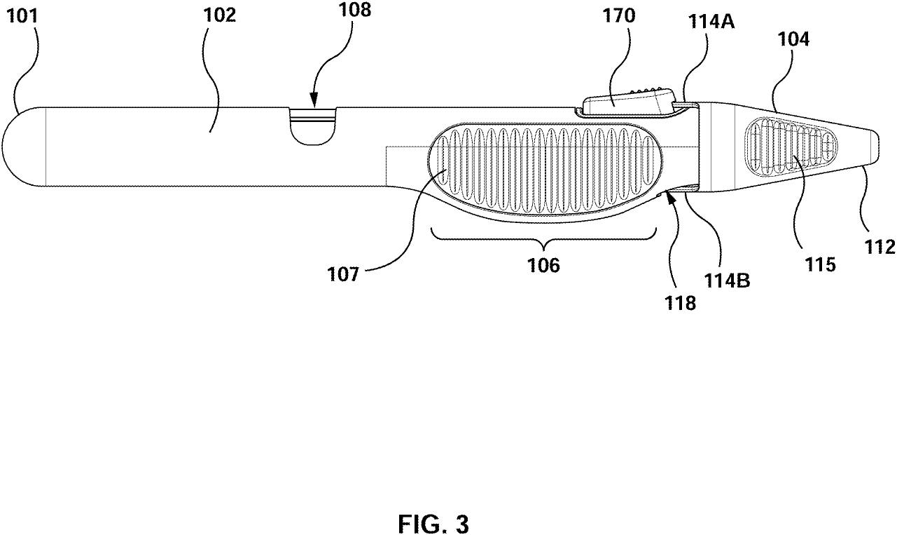

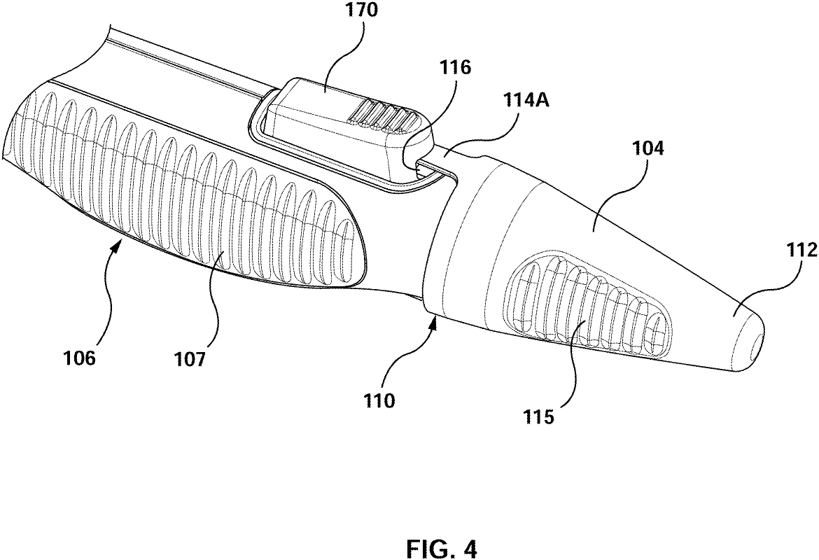

FIGS. 3 and 4 illustrate the safety cap 104 coupled to the housing 102 of the injector, with FIG. 4 being an enlarged perspective view of the distal end portion of the injector 100. The safety cap 104 is configured to be removably coupled to the housing 102. The safety cap 104 covers the distal end 123 of the cannula 122 when the injector 100 is not in use and is configured so that the actuator 170 cannot be actuated when the safety cap 104 is coupled to the housing 102 in order to prevent inadvertent actuation of the injector 100. More particularly, as shown in FIGS. 3 and 4, when coupled to the housing 102 the safety cap 104 covers and protects the distal end portion of the injector 100, including the distal end 123 of the cannula 122 which is not disposed within the housing 102. The safety cap 104 has a proximal end 110 that is configured to couple to the distal end 103 of the housing 102. The safety cap 104 has a generally conical configuration, tapering from the proximal end 110 to a distal tip 112. The safety cap 104 further includes a finger-gripping surface 115 thereon. Although FIGS. 3 and 4 show only one side of the safety cap 104, a similar finger-gripping surface is preferably included on the opposing side of the safety cap 104 to permit a user to easily hold and grip the safety cap 104 during removal thereof. Each finger-gripping surface 115 may be formed from an elastomeric material and may include a plurality of ribs formed thereon. Any of a variety of shapes or configurations may be selected for the finger-gripping surface 115 to provide suitable finger placement during injector operation.

The proximal end 110 of the safety cap 104 includes a pair of opposing tabs 114A, 114B. When the safety cap 104 is coupled to the housing 102, the tab 114A extends into a slot or opening 116 formed through the actuator 170 in order to prevent inadvertent actuation of the actuator 170. Stated another way, when the tab 114A is disposed within the slot 116, the actuator 170 cannot be depressed or actuated by a user. Thus, the actuator 170 is configured to interface with the safety cap 104 such that the injector 100 cannot be operated when the safety cap 104 is coupled to the housing 102. When the safety cap 104 is coupled to the housing 102, the tab 114B extends into a slot or opening 118 formed through the asymmetrical fin 106 of the housing 102 in order to further secure the safety cap 104 onto the housing 102.

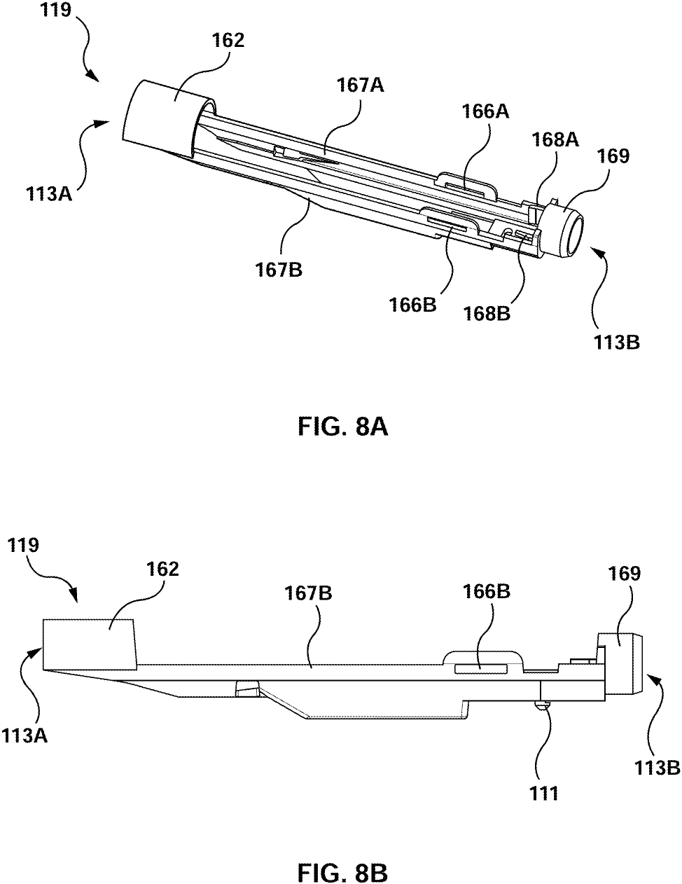

FIGS. 8A and 8B are perspective and side views, respectively, of a window chassis 119 of the injector 100, and the window chassis 119 is shown removed from the injector 100 for sake of illustration. The window chassis 119 is a clear component formed from a relatively hard material such as polycarbonate or acrylic. The window chassis 119 is fixed or secured to the housing 102 and does not move relative thereto. In another embodiment hereof (not shown), the features or functionalities of the window chassis 119 may be integrated or built into the housing 102 such that the window chassis 119 may be eliminated. The window chassis 119 includes a proximal end 113A and a distal end 113B. At the proximal end 113A, the window chassis 119 includes a semi-circular portion 162. When the chassis 119 is secured within the housing 102, the window 108 of the housing 102 is disposed over semi-circular portion 162 of the window chassis 119. Since the window chassis 119 is formed from a clear material, it permits visualization of the shuttle subassembly 150 during operation of the injector 100 as described above.

The window chassis 119 is configured to receive the magazine subassembly 130 therein. At the distal end 113B, the window chassis 119 includes an annular ring portion 169. Proximal to the distal end 113B, the window chassis 119 includes a pair of integral ledges 168A, 168B formed on an inner surface thereof for receiving corresponding features (prongs 129A, 129B that will be described in more detail herein) of the magazine subassembly 150 to secure the magazine subassembly 150 to the window chassis 119.

The window chassis 119 is also configured to receive the shuttle subassembly 150 slidingly therein. Stated another, the shuttle subassembly 150 slides or moves relative to the window chassis 119 during operation of the injector 100. The window chassis 119 includes a pair of opposing rails 167A, 167B. The shuttle subassembly 150 slides or moves within the window chassis 119 along the rails 167A, 167B during operation of the injector 100. Further, near the distal end 113B, the window chassis 119 further includes a hook 111 formed on an outer surface thereof. A spring 156 of the shuttle assembly 150 is attached to the hook 111 as will be described in more detail below.

The window chassis 119 is also configured to be secured to an actuator chassis 172 that receives and interacts with the actuator 170 as will be described in more detail herein. More particularly, the window chassis 119 includes a pair of openings or slots 166A, 166B for receiving corresponding tabs 141A, 141B of the actuator chassis 172 in a snap-fit arrangement to secure the actuator chassis 172 to the window chassis 119.

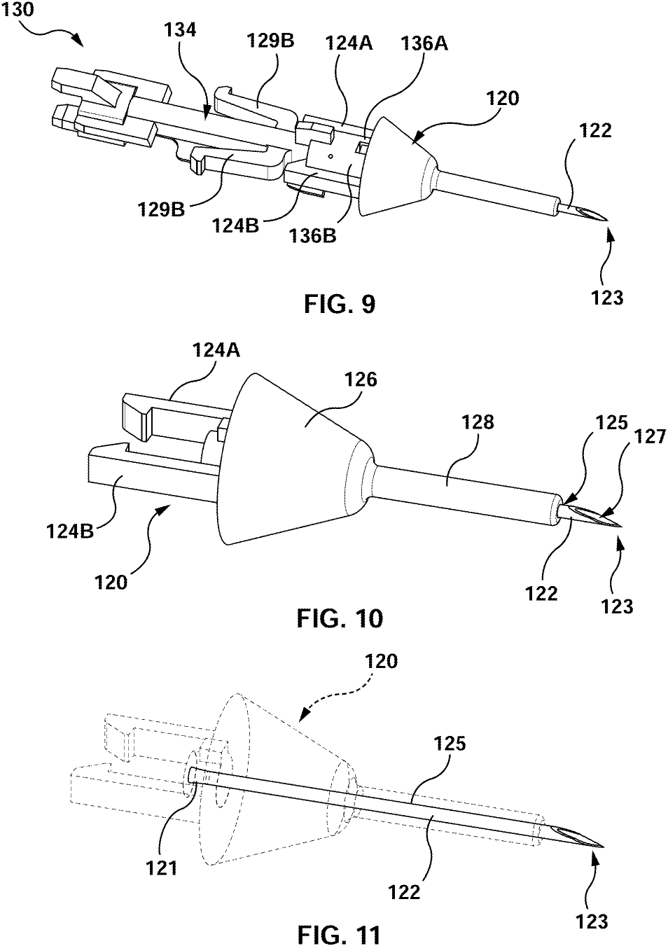

With reference to FIGS. 9-17, the magazine subassembly 130 of the injector 100 will now be described in more detail. FIG. 9 is a perspective view of the magazine subassembly 130 of the injector 100. In addition to the cannula 122, the magazine tube 132, and the gate 140, the magazine subassembly 130 also includes a cannula mount 120 and a magazine tube mount 134. The cannula 122 is securely attached to and disposed within the cannula mount 120, and the magazine tube 132 is securely attached to and disposed within the magazine tube mount 134. The cannula mount 120 is secured via a snap fit attachment to the magazine tube mount 134, as shown in FIG. 9. In the configuration of FIG. 9, the magazine subassembly 130 may be the last component that is inserted or slid into the housing 102 of the injector 100 for final assembly thereof. When inserted into the housing 102, the proximal end of the magazine subassembly 130 (including prongs 129A, 129B that will be described in more detail herein) is configured to snap onto the integral ledges 168A, 168B of the window chassis 119. After being secured to the window chassis 119, the magazine subassembly 130 is fixed relative to the housing 102 and relative to the window chassis 119 and does not move relative thereto.

FIG. 10 is a perspective view of the cannula mount 120 and the cannula 122 secured thereto, and FIG. 11 is a similar view showing the cannula mount in phantom for illustrative purposes. The cannula mount 120 includes a pair of opposing prongs 124A, 124B at a proximal end thereof for coupling the cannula mount 120 to the magazine tube mount 134 via a snap fit attachment. The cannula mount 120 also includes a conical portion 126 and a tubular portion 128 at a distal end thereof. The conical portion 126 and the tubular portion 128 include a continuous lumen 125 therethrough for receiving the cannula 122. When assembled into the injector 100, the conical portion 126 of the cannula mount 120 abuts against the distal end 103 of the housing 102. The tubular portion 128 of the cannula mount 120 distally extends from the conical portion 126 and supports a portion of the cannula 122 that extends outside of the housing 102.

The cannula 122 is fixed or secured within the continuous lumen 125 of the cannula mount 120 so that the cannula 122 does not move relative to the cannula mount 120. The cannula 122 has a lumen 127 extending the entire length thereof, from a proximal end or inlet 121 to the distal end 123, which may also be considered the outlet of the cannula 122. The lumen 127 is sized or configured to slidingly receive the push rod 160 therethrough. As best shown in FIG. 11, the proximal end 121 of the cannula 122 may have a flared configuration, with an outer diameter greater than the remaining length of the cannula 122, in order to further prevent dislodgement or any distal movement of the cannula 122 within the cannula mount 120 when the push rod 160 is advanced through the cannula 122. When assembled into the injector 100, the proximal end 121 of the cannula 122 is disposed within the housing 102.

The distal end 123 of the cannula 122 is beveled and is configured to be inserted into an eye. In an embodiment, the bevel of the distal end 123 is oriented upwards such that the bevel of the distal end 123 is aligned with the actuator 170 of the injector. As best shown in FIG. 10, the distal end 123 extends distally beyond a distal end of the tubular portion 128 of the cannula mount 120, so that the distal end 123 is exposed for insertion into the eye.

The cannula 122 may be formed from tubing between 18-gauge and 30-gauge that is adapted to penetrate a sclera of an eye. Although the cannula 122 preferably has a straight longitudinal profile, other suitable longitudinal needle shapes may be used. The bevel of the distal end 123 may disposed at an angle of about between 10 and 13 degrees, preferably about 11.5 degrees, in relation to the longitudinal axis of the cannula 122. The cannula 122 may be made of any suitably rigid material such as metal or metal alloys, for example stainless steel, or a polymeric material such as polyimide, silicone, polycarbonate and/or polyvinyl carbonate. The cannula 122 may have an external diameter between 0.25 mm and 1.0 mm.

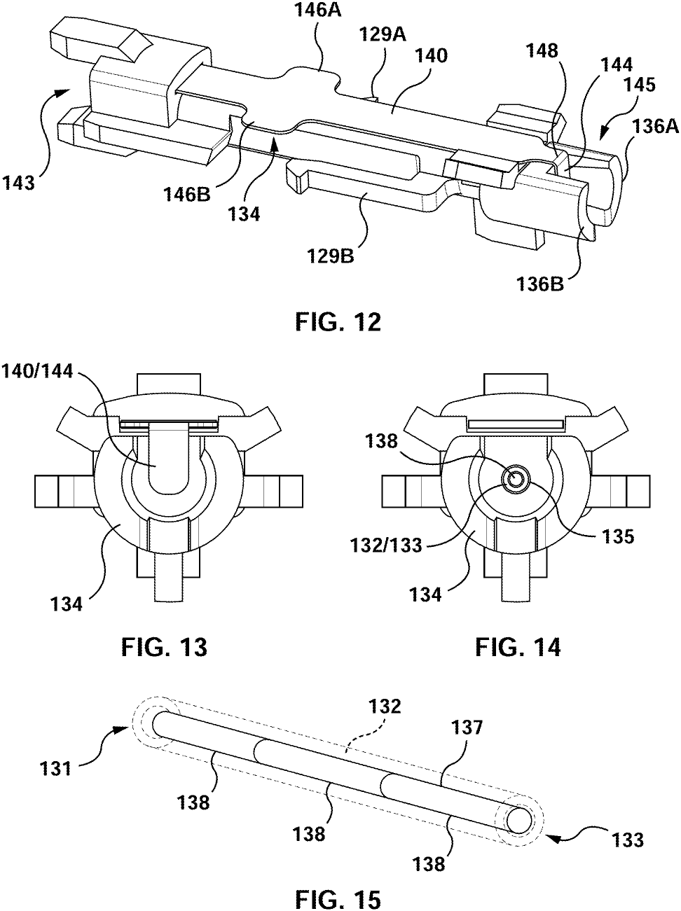

Perspective and end views of the magazine tube 132, the magazine tube mount 134, and the gate 140 are shown in FIGS. 12-13. Further, FIG. 14 is the same end view as FIG. 13 except that the gate 140 is removed for sake of illustration. The magazine tube mount 134 includes a proximal end 143 and a distal end 145. The distal end 145 of the magazine tube mount 134 includes two opposing, semi-circular tabs 136A, 136B. The pair of opposing prongs 124A, 124B of the cannula mount 120 grasp or hook in a snap-fit arrangement onto the semi-circular tabs 136A, 136B of the magazine tube mount 134 to attach the cannula mount 120 to the magazine tube mount 134, as best shown on FIG. 9. The magazine tube mount 134 includes a pair of opposing prongs 129A, 129B for coupling the magazine tube mount 134 to the window chassis 119 via a snap-fit arrangement. The magazine tube mount 134 includes a continuous lumen 135 therethrough for receiving the magazine tube 132.

The magazine tube 132 is fixed or secured within the lumen 135 of the magazine tube mount 134 so that the magazine tube 132 does not move relative to the magazine tube mount 134. The magazine tube 132 has a lumen 137 extending the entire length thereof, from a proximal end or inlet 131 to a distal end or outlet 133. The lumen 137 is sized or configured to slidingly receive the push rod 160 therethrough. In addition, as best shown in FIG. 15 which is a perspective view of the magazine tube 132 removed from the magazine tube mount 134 for illustrative purposes and shown in phantom, the magazine tube 132 is sized and configured to hold or retain up to three implants 138 in a serial arrangement when the injector 100 is in the non-deployed state. Stated another way, prior to operation of the injector 100, the implants 138 reside within the lumen 137 of the magazine tube 132. The injector 100 is configured to consecutively deliver the three implants 138 to an eye via a single actuation of the actuator 170.

In an embodiment, the implants 138 may be pre-loaded into the magazine subassembly 130 by a drug manufacturer. More particularly, the implants 138 may be pre-loaded into the magazine tube 132 of the magazine subassembly 130 prior to assembly of the injector, and the drug manufacturer may store and/or ship the magazine subassembly 130 having the implants 138 pre-loaded therein. After shipment, the magazine subassembly 130 having the implants 138 pre-loaded therein may be inserted into the housing 102 for final assembly of the injector 100 prior to use. As such, the magazine subassembly 130 may be considered a single use, sterilizable cartridge that may be manufactured and shipped separately from the remaining components of the injector 100. During shipment, the inlet 131 of the magazine tube 132 may be plugged for delivery and the outlet 133 of the magazine tube 132 is blocked or occluded via the gate 140 as will be described in more detail herein.

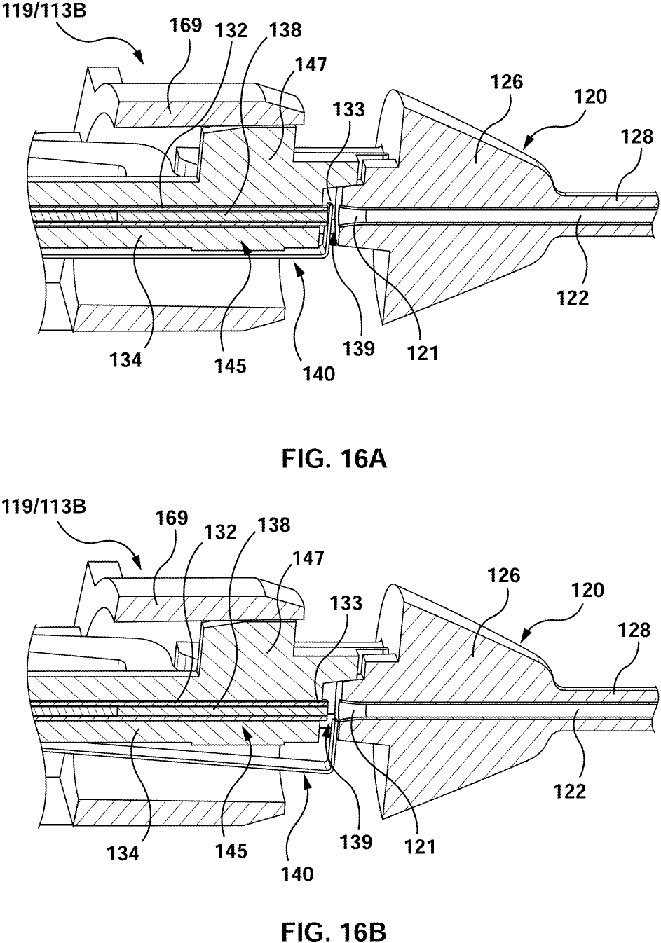

With reference to FIG. 16A, which is enlarged sectional view of the magazine subassembly 130 when the injector 100 is in the non-deployed state and the gate 140 is in a closed configuration, the cannula 122 and the magazine tube 132 are coaxially aligned. Notably, the magazine subassembly 130 is concentrically located within the window chassis 119 when secured therein. At the distal end 145 thereof, the magazine mount 134 includes a plurality of radially-extending ribs 147 to limit or restrict movement of the magazine subassembly 130 within the window chassis 119. When assembled into the window chassis 119, the plurality of radially-extending ribs 147 are disposed within the annular ring portion 169 at the distal end 113B of the window chassis 119 as shown in FIG. 16A.

As shown in FIG. 16A, the distal end or outlet 133 of the magazine tube 132 is spaced apart from the proximal end or inlet 121 of the cannula 122 by a transition gap 139. When the gate 140 is disposed within the transition gap 139, as shown in FIG. 16A, the gate 140 covers or blocks the distal end or outlet 133 of the magazine tube 132. Stated another way, when the gate 140 is disposed within the transition gap 139, the gate 140 does not permit the lumen 137 of the magazine tube 132 to be in fluid communication with the lumen 127 of the cannula 122.

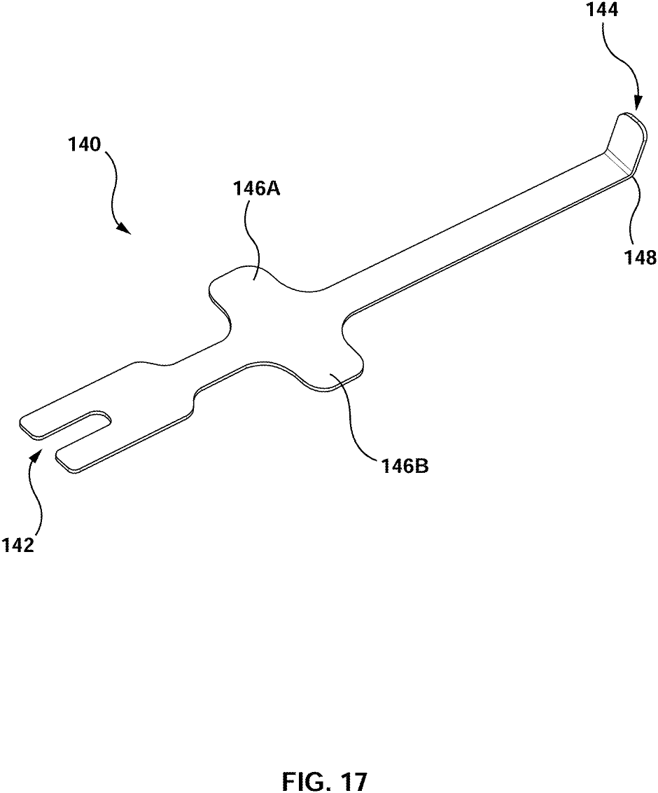

FIG. 17 is a perspective view of the gate 140 removed from the magazine tube mount 134. The gate 140 is a generally planar component formed from sheet metal that includes a proximal end 142, a distal end 144, and a pair of lateral wings 146A, 146B. In an embodiment, a thickness of the gate 140 along a length thereof may vary such that portions thereof that flex or deflect during operation of the injector 100 are relatively thinner or thinned out to minimize the force required for movement. The proximal end 142 of the gate 140 is secured and fixed to the proximal end 143 of the magazine tube mount 134, as shown in FIG. 12. The remaining length of the gate 140 is not fixed to the magazine and may be displaced therefrom. The gate 140 includes an integral bend 148, which is approximately 90 degrees, so that the distal end 144 extends along a perpendicular plane relative to the remaining length of the gate 140 during operation of the injector 100. When the injector 100 is in the non-deployed state, most of the gate 140 abuts against or extends alongside an underside surface of the magazine tube mount 134 except the distal end 144 of the gate 140, which extends over the distal end or outlet 133 of the magazine tube 132 as best shown in FIG. 12, FIG. 13 or FIG. 16A. The gate 140 may be considered to be in a closed configuration when the distal end 144 thereof covers or extends over the distal end or outlet 133 of the magazine tube 132. The cannula 122 is not in fluid communication with the magazine tube 132 when the gate 140 is in the closed configuration, because the distal end 144 of the gate 140 is disposed between the magazine tube 132 and the cannula 122 within the transition gap 139.

Actuation of the actuator 170 from a non-deployed position to a deployed position (which will be described in more detail below) moves or displaces the gate 140 from the closed configuration to an open configuration. FIG. 16B is an enlarged sectional view of the magazine subassembly 130 with the gate 140 in the open configuration. The gate 140 may be considered to be in the open configuration when the distal end 144 thereof does not cover or extend over the distal end or outlet 133 of the magazine tube 132 such that the outlet 133 of the magazine tube 132 is exposed or open. Further, the cannula 122 is in fluid communication with the magazine tube 132 when the gate 140 is in the open configuration. When the actuator 170 is actuated, the actuator 170 contacts the lateral wings 146A, 146B of the gate 140 to push or move the gate 140 downwards in a direction away from the underside surface of the magazine tube mount 134. The gate 140 is configured to deflect or flex when the actuator 170 applies a sufficient force thereto to transition to the open configuration.

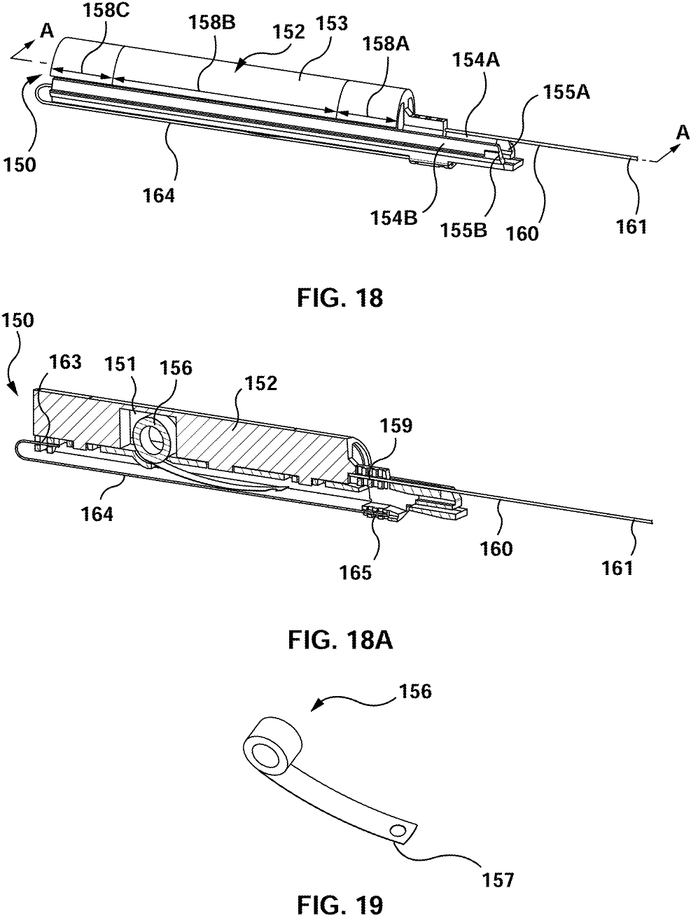

Turning now to FIGS. 18-19, the shuttle subassembly 150 will be described in more detail. FIG. 18 is a perspective view of the shuttle subassembly 150 removed from the injector 100 for sake of illustration. FIG. 18A is a sectional view taken along line A-A of FIG. 18. The shuttle subassembly 150 includes a shuttle body 152, the spring 156, the pushrod 160, and a drag wire 164. The shuttle body 152 includes a cavity 151 formed therein for housing the spring 156. The shuttle body 152 also includes a pair of distally-extending fingers 154A, 154B at a distal portion thereof. Each distally-extending finger 154A, 154B includes a distal end surface 155A, 155B for interacting with the actuator 170 as will be described in more detail herein.

A proximal end 159 of the push rod 160 is fixed or secured to the shuttle body 152 as shown in FIG. 18A so that the push rod 160 moves or translates with the shuttle body 152. The proximal end 159 of the push rod 160 may be fixed or secured to the shuttle body 152 via adhesive or welding, or other any suitable mechanical method. In an embodiment, the proximal end 159 of the push rod 160 is secured to the shuttle body 152 via both adhesive and a mechanical interlock. When assembled into the injector 100, the push rod 160 is coaxially aligned or concentric with each of the magazine tube 132 and the cannula 122, and is sized to be slidingly received within the lumens 127, 137, of the magazine tube 132 and the cannula 122, respectively. When the shuttle body 152 moves distally, i.e., in a direction towards the cannula 122, the push rod 152 is configured to enter the lumens 127, 137, of the magazine tube 132 and the cannula 122, respectively, to push or distally advance the implants 138 from the magazine tube 132, into and through the cannula 122, and ultimately ejected through the outlet or distal end 123 of the cannula 122 into the eye. The push rod 160 has a straight longitudinal profile and may be made of any suitably rigid material such as metal or metal alloys, for example stainless steel. When in the non-deployed state of the injector 100, a distal end 161 of the push rod 160 is disposed proximal to the proximal end or inlet 131 of the magazine tube 132. In an embodiment, the distal end 161 of the push rod 160 is configured to be slightly spaced apart from the inlet 131 of the magazine tube 132 prior to deployment to ensure that no force or load is applied to the implants 138 prior to operation of the injector 100. After deployment, in the deployed state of the injector 100, the distal end 161 of the push rod 160 is disposed distal to the distal end 123 of the cannula 122 as can be seen in FIGS. 6 and 7.

FIG. 19 illustrates the spring 156 removed from the shuttle assembly 150 for sake of illustration only. The shuttle body 152 may be constructed from two halves, thereby forming the cavity 151. The spring 156 is housed or disposed within the cavity 151 of the shuttle body 152. The spring 156 is a constant force spring that is biased or shape-set into a coiled, or non-extended, configuration. When assembled within the injector 100 in the non-deployed state, the spring 156 is stretched into an extended configuration. When in the extended configuration, at least a portion of the spring 156 is elongated and non-coiled, and a tail or free end 157 thereof is attached to the hook 111 of the window chassis 119. Prior to deployment or operation of the injector 100, as will be explained in more detail herein, the actuator 170 is coupled to the shuttle body 152 and is configured to hold or retain the shuttle body 152 in a first position in which the spring 156 is stretched into the extended configuration. When the actuator 170 is actuated, the actuator 170 releases or decouples from the shuttle body 152 and the spring 156 is permitted to resume its coiled, or non-extended configuration, due to the biased or shape-set nature thereof. Since the tail or free end 157 of the spring 156 is attached to the hook 111 of the window chassis 119, coiling of the spring 156 moves or translates the shuttle body 152 and the push rod 160 in a distal direction.

The shuttle body 152 includes an outer surface 153 that may include status indicators disposed or formed thereon to alert the user of the relative positioning of the shuttle subassembly 150 within the housing 102. As described above, the housing 102 includes the window 108 formed therein so that a user can track the axial movement or translation of the shuttle subassembly 150 within the housing 102. Thus, a user can see movement of the shuttle subassembly 150 during operator of the injector 100. For example, a distal segment 158A of the outer surface 153 may include a first status indicator, a middle segment 158B of the outer surface 153 may include a second status indicator, and a proximal segment 158C of the outer surface 153 may include a third status indicator. Prior to deployment or actuation of the actuator 170, for example, a user will see the first status indicator of the distal segment 158A displayed through the window 108 of the housing 102 to provide visual feedback to the user that the deployment operation has not yet been initiated. As the shuttle subassembly 150 is moving distally from its initial position to its final position within the housing 102, a user will see the second status indicator of the middle segment 158B displayed through the window 108 of the housing 102 to provide visual feedback to the user that the shuttle subassembly 150 is moving and deployment is underway. When movement of the shuttle subassembly 150 is complete and the shuttle assembly is at its final position within the housing 102, a user will see the third status indicator of the proximal segment 158C displayed through the window 108 of the housing 102 to provide visual feedback to the user that the deployment operation is complete. As previously mentioned, the status indicators may include notations, symbols, instructions, or colors. In an embodiment, the first status indicator may be green to indicate that the injector 100 is ready for operation, the second status indicator may be yellow to indicate that deployment of the injector 100 is underway, and the third status indicator may be red to indicate deployment of the injector 100 is complete.

In an embodiment hereof, the injector 100 is configured such that a speed of delivery of the implants 138 is predetermined or controlled and the speed of delivery is between 2 and 12 seconds. In an embodiment hereof, the speed of delivery is between 3 and 10 seconds, or approximately 6.5 seconds with approximately defined as a tolerance of 3.5 seconds. In an embodiment hereof, the speed of delivery is between 5 and 7 seconds, or approximately 6 seconds with approximately defined as a tolerance of 1 second. Controlling the speed of delivery is important for several reasons. Since the injector 100 is configured to deliver up to three implants 138, each implant must be ejected from the injector 100 sequentially. When the application or target site is within tissue of the eye, the implants 138 may tend to exit from the injector 100 in a substantially straight trajectory, towards the back of the eye. Thus, the length or amount that the implants may travel is limited or restricted due to anatomy constraints. If the implants 138 are ejected too fast or quickly, one or more implants 138 may contact and damage the back of the eye. However, it is also desirable to minimize the overall operation time because the patient cannot move while the device is being operated. The injector 100 and dispensing procedure, including the overall operation time, should minimize patient discomfort and avoid injury. Accordingly, the injector 100 includes means for controlling the speed of delivery of the implants 138. The time ranges described above refer to the time period that elapses from full activation of the actuator 170 to when all three implants 138 have been ejected from the injector 100 and thus deployment is complete. For example, in an embodiment when the speed of delivery is between 3 and 10 seconds, the injector 100 dispenses the full dose of implants 138 in no less than 3.0 seconds, starting from the time of full activation of the actuator 170 to the time the trailing end of the last implant clears the distal end 123 of the cannula 122. Further, the total time from full activation of the actuator 170 to when the trailing end of edge of the last implant 138 clears the distal end 123 of the cannula 122 is no greater than 10.0 seconds. A three second dispensing time is slow enough to allow the implants 138 to drop into the vitreous to avoid injury to the patient’s eye as well as avoid damaging the implants 138. Conversely, a total time of ten seconds is considered fast enough to support safe insertion, dispensing, and removal of the cannula 122 from the eye. In addition, the implants 138 are preferably delivered at a constant rate such that one implant does not dart out while the others are slow enough to meet the total delivery speed requirement. In an embodiment, the injector 100 dispenses the full dose of implants 138 at a constant rate or speed such that the time for the fastest implant is no more than 20% faster than the time for the slowest implant.

More particularly, in an embodiment, the shuttle subassembly 150 further includes a drag wire 164 and a shuttle decelerator 190 which interact with each other to control the speed of delivery of the implants 138 as described above. The drag wire 164 is secured to the shuttle body 152 so that the drag wire 164 moves or translates therewith. As shown in FIG. 18A, a proximal or first end 163 of the drag wire 164 is attached or fixed to a proximal portion of the shuttle body 152, and a distal or second end 165 of the drag wire 164 is attached or fixed to a distal portion of the shuttle body 152. In an embodiment, the proximal and distal ends 163, 165 of the drag wire 164 are secured to the shuttle body 152 via both adhesive and a mechanical interlock. The drag wire 164 is disposed within the housing 102 and extends adjacent to or alongside an underside surface of the shuttle body 152. As will be described in more detail herein, the drag wire 164 interacts with the shuttle decelerator 190 to slow down deployment of the shuttle body 152 and push rod 160. The drag wire 164 may be formed from metal or metal alloys, for example stainless steel.

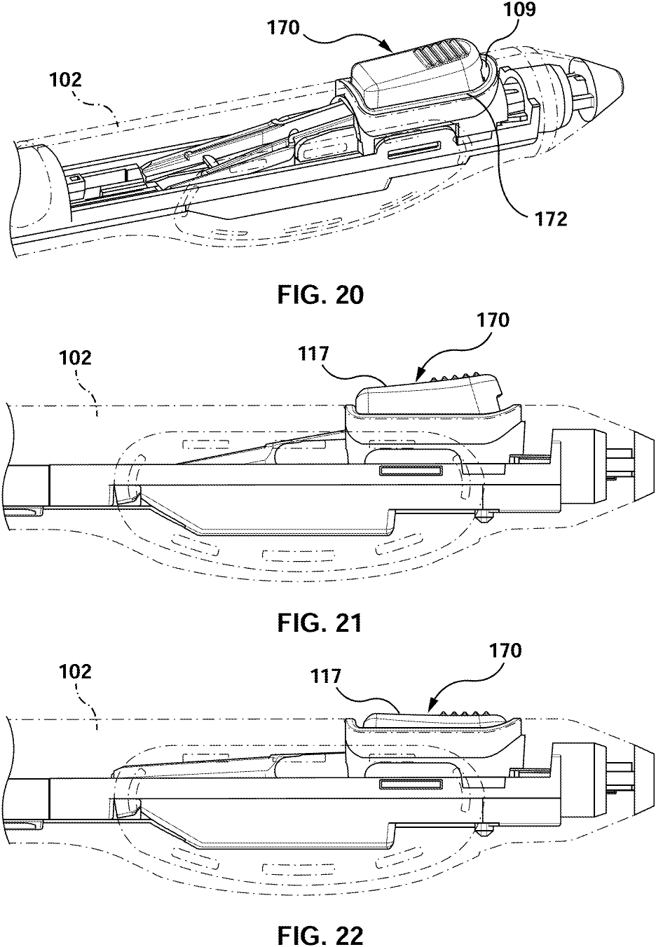

The actuator 170 will now be described in more detail with reference to FIGS. 20-29. FIGS. 20 and 21 are enlarged perspective and side views, respectively, of the actuator 170 when the injector 100 is in the non-deployed state, while FIG. 22 is an enlarged side view of the actuator 170 when the injector 100 is in the deployed state. The actuator 170 extends through the opening 109 within the housing 102, and is accessible to a user to operate the injector 100 from the non-deployed state of FIGS. 20-21 to the deployed state of FIG. 22. When in a non-deployed position, the actuator 170 protrudes outwardly from the housing 102 and an outer surface 117 of the actuator 170 forms an acute angle between 10-25 degrees relative to a longitudinal axis of the injector 100, as best shown in FIG. 21. To actuate the actuator 170, a user presses the actuator 170 downward, in a direction towards the housing 102. As a result, the actuator 170 will travel or be displaced downward approximately 4 mm and will also pivot within the housing 102 as will be explained in more detail herein. When in a deployed position, the outer surface 117 of the actuator 170 is substantially parallel with the longitudinal axis of the injector 100 and further is approximately flush with an outer surface of the housing 102, as best shown in FIG. 22.

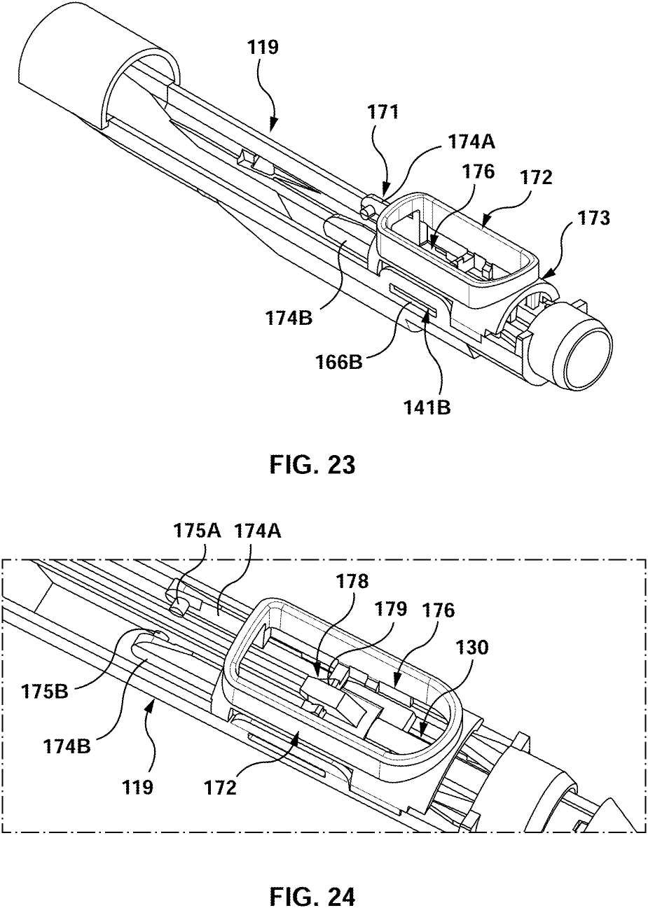

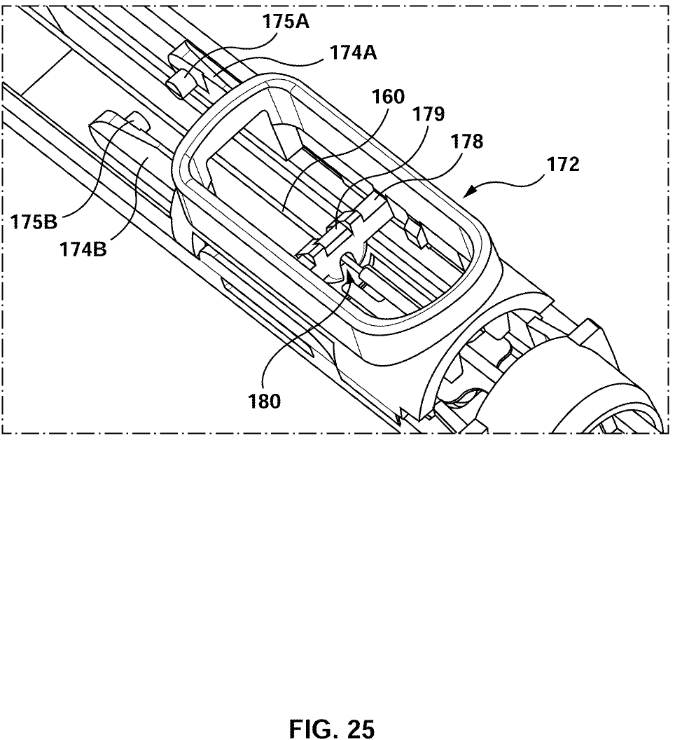

The actuator 170 is mounted within an actuator chassis 172. With reference to FIG. 23, the actuator chassis 172 is fixed or secured within the window chassis 119 so that the actuator chassis 172 does not move relative to the window chassis 119. The actuator chassis 172 includes a proximal end 171, which includes a pair of prongs 174A, 174B with inwardly-extending posts 175A, 175B, respectively, for coupling to the actuator 170, and a distal end 173. The inwardly-extending posts 175A, 175B are best shown on FIGS. 24 and 25. The actuator chassis 172 further includes a pair of tabs 141A, 141B for securing the actuator chassis 172 to the window chassis 119. Tab 141A is not visible on FIG. 23, but is disposed on an opposing location on the actuator chassis 172. The tabs 141A, 141B are configured to be received within slots 166A, 166B of the window chassis 119 in a snap-fit arrangement. The actuator chassis 172 defines a generally rectangular opening 176 for receiving the actuator 170 therein.

With reference to FIG. 24, which illustrates the coupling of the magazine subassembly 130 to the actuator chassis 172, the actuator chassis 172 includes a support beam 178 extending across the opening 176. The support beam 178 defines an opening 179 that is configured to receive a mating snap fit feature of the magazine subassembly 130. When the magazine subassembly 130 is slid into the housing 102 for final assembly of the injector 102, the snap fit feature thereof snaps into the opening 179 to secure or fix the magazine assembly 130 relative to the actuator chassis 172. During operation of the injector 170, the window chassis 119, the actuator chassis 172, and the magazine subassembly 130 (except for gate 140) are stationary components that do not move relative to each other.

In addition to supporting the magazine subassembly 170, the support beam 178 of the actuator chassis 172 also includes a channel 180 formed therethrough for receiving the push rod 160 as shown in FIG. 25. The channel 180 functions to coaxially locate the push rod 160 relative to the magazine tube 132 and the cannula 122. As described above, the push rod 160 is coaxially aligned with each of the magazine tube 132 and the cannula 122 and is sized to be slidingly received within the lumens 127, 137, of the magazine tube 132 and the cannula 122, respectively.

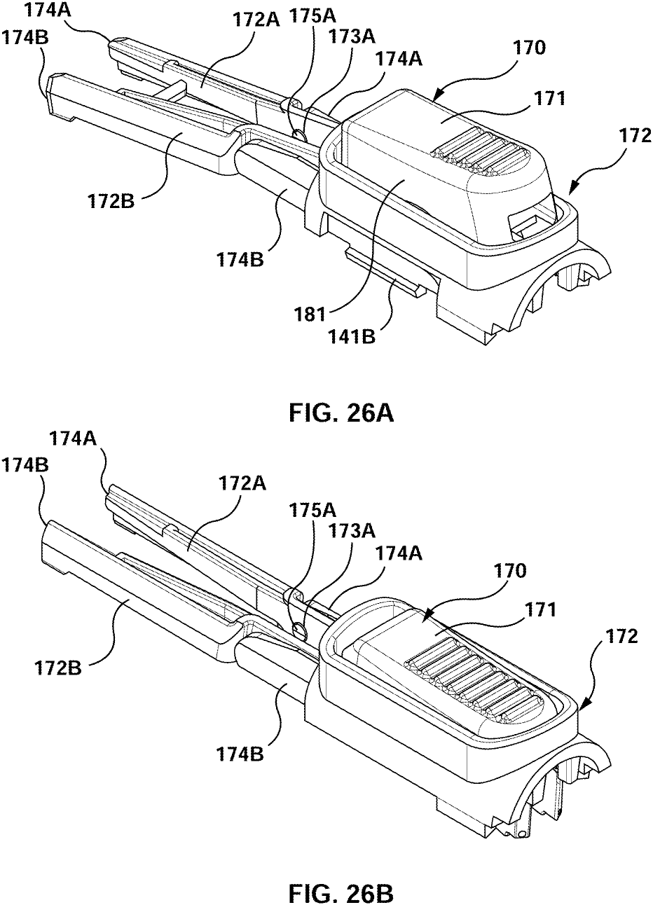

FIGS. 26A and 26B illustrate the pivoting movement of the actuator 170 relative to the actuator chassis 172 as the actuator 170 is actuated from the non-deployed position to the deployed position. Particularly, FIG. 26A shows the relative positioning of the actuator 170 when the actuator 170 is in the non-deployed position while FIG. 26B shows the relative positioning of the actuator 170 when the actuator 170 is in the deployed position. The actuator 170 includes a body portion 181, which includes the outer surface 117 thereon, and the body portion 181 is sized and configured to be received within the opening 176 of the actuator chassis 172. The actuator 170 also includes a pair of proximally-extending arms 172A, 172B that are integrally formed with the body portion 181. The proximally-extending arms 172A, 172B define proximal end surfaces 174A, 174B, respectively, that interact with and detachably couple to the shuttle subassembly 150 as will be described in more detail herein. The proximally-extending arms 172A, 172B also includes openings or holes 173A, 173B, respectively, that are configured to receive inwardly-extending posts 175A, 175B, of prongs 174A, 174B, respectively, of the actuator chassis 172. Opening or hole 173B is not visible on FIGS. 26A and 26B, but is disposed on an opposing location on the proximally-extending arm 172B of the actuator 170. The coupling between the inwardly-extending posts 175A, 175B and the holes 173A, 173B, respectively, permits the actuator 170 to pivot or rotate relative to the actuator chassis 172.

When the actuator 170 is in the non-deployed position, as shown in FIG. 26A, the proximally-extending arms 172A, 172B of the actuator 170 are angled downward, i.e., angled with respect to the parallel to the longitudinal axis of the injector 100, and the proximal end surfaces 174A, 174B contact and abut against the distal end surfaces 155A, 155B, respectively, of the shuttle subassembly 150. When the actuator 170 is pressed downward by a user for actuation, the actuator 170 travels or is displaced downward and also pivots within the actuator chassis 172 around the inwardly-extending posts 175A, 175B of the actuator chassis 172. Stated another way, the inwardly-extending posts 175A, 175B function as a fulcrum on which the actuator 170 pivots.

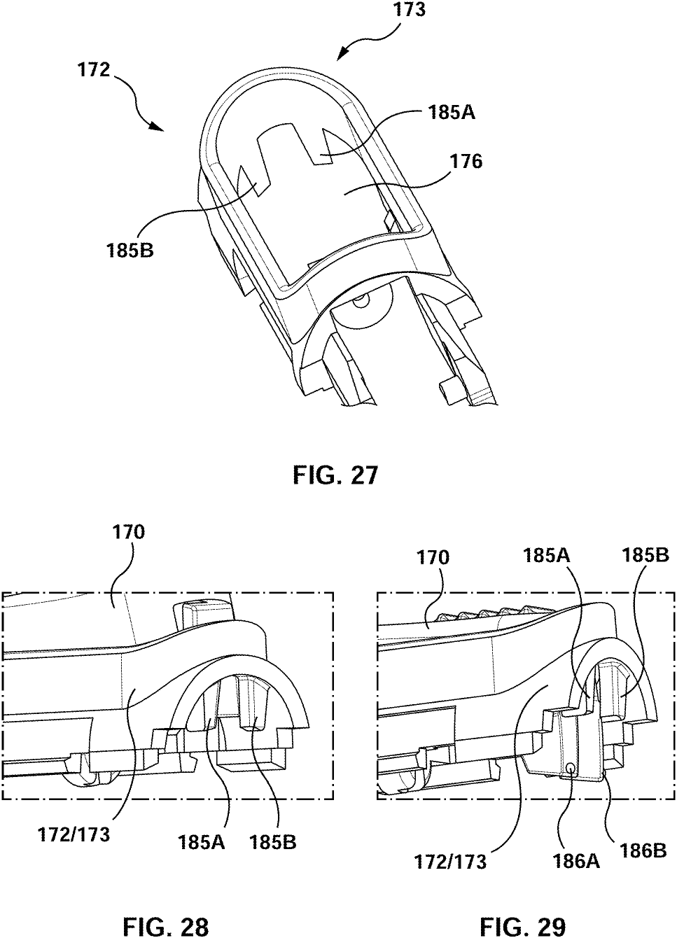

When the actuator 170 is in the deployed position, as shown in FIG. 27A, the proximally-extending arms 172A, 172B of the actuator 170 extend approximately parallel to the longitudinal axis of the injector 100. Further, the proximally-extending arms 172A, 172B of the actuator 170 also extend approximately parallel to the distally-extending fingers 154A, 154B of the shuttle subassembly 150 and are spaced apart therefrom, such that the proximal end surfaces 174A, 174B of the actuator 170 no longer contact or abut against the distal end surfaces 155A, 155B, respectively, of the shuttle subassembly 150. When the actuator 170 is in the deployed position, the shuttle subassembly 150 is effectively decoupled or released from the actuator 170 and the shuttle subassembly 150 is free to be moved or translated by the spring 156 as described above.

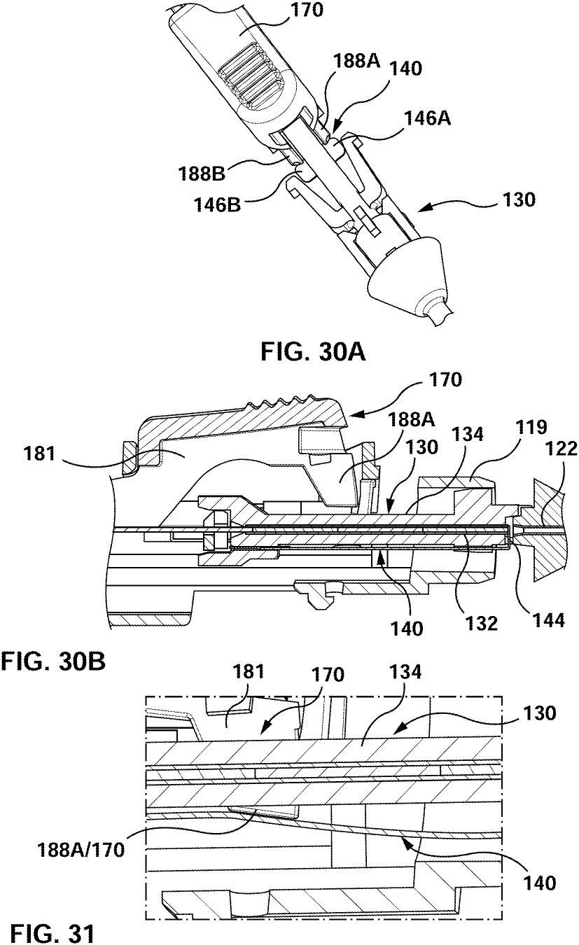

In order to increase the resistance of pressing the actuator 170 downward for actuation, the actuator chassis 172 may include a pair of teeth 185A, 185B disposed at the distal end 173 of the actuator 170 as shown on FIG. 27. The teeth 185A, 185B extend downward, i.e., in a direction towards an interior of the housing 102, and an angled radially inwards towards a center of the opening 176 of the actuator chassis 172. The actuator 170 includes a pair of knobs 186A, 186B formed on a pair of legs 188A, 188B (see FIG. 29 and FIG. 30A) that extend from the body portion 181 of the actuator 170. The knobs 186A, 186B are configured and positioned to contact and press against the teeth 185A, 185B, respectively, in an interference fit when the actuator 170 is in the non-deployed position, as shown in FIG. 28. When the actuator 170 is pressed downward during actuation thereof to the deployed position as shown in FIG. 29, the actuation force must be sufficient to force the pair of knobs 186A, 186B out of the interference fit with the teeth 185A, 185B in order to depress the actuator 170. Thus, the teeth 185A, 185B function to increase the minimal actuation force required to actuate the actuator 170 so that actuator 170 is not overly sensitive to actuation, which may lead to inadvertent deployment.

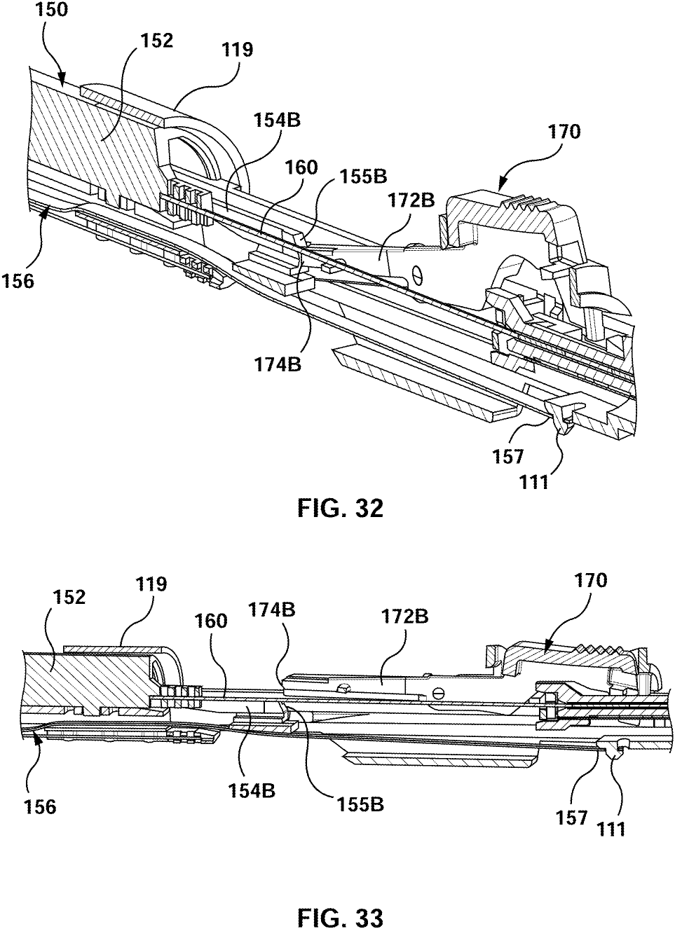

With the structure of the actuator 170 described above, the operation of the actuator 170 will now be described in more detail with respect to FIGS. 30A-33. Movement of the actuator 170 from the non-deployed position to the deployed position results has two stages of actuation: (1) the actuator 170 moves or displaces the gate 140 from the closed configuration to the open configuration, and (2) the actuator 170 decouples from and releases the shuttle subassembly 150, thereby causing movement or translation of the pushrod 160 into and through the magazine tube 132 and the cannula 122 in order to deliver the at least one implant contained within the magazine tube 132 through the cannula 122 and into the eye. The first stage of actuation in which the actuator 170 moves or displaces the gate 140 from the closed configuration to the open configuration is shown in FIGS. 30-31, while the second stage of actuation in which the actuator 170 decouples from and releases the shuttle subassembly 150 is shown in FIGS. 32-33. When the actuator 170 is depressed by a user, the first stage of actuation occurs before the second stage of actuation. Initial depression of the actuator 170 by a user causes the actuator 170 to move or displace the gate 140 from the closed configuration to the open configuration, and the actuator subsequently decouples from and releases the shuttle subassembly 150 when the actuator 170 is further or completely pressed down by the user. Thus, the gate 140 is moved to the open configuration before the shuttle subassembly 150 (including the push rod 160) is released.

The first stage of actuation is shown in FIGS. 30A, 30B and 31. More particularly, FIG. 30A is a perspective view of the actuator 170 and the magazine subassembly 130 and FIG. 30B is an enlarged sectional view of the injector 100. FIGS. 30A and 30B illustrate the relative positioning of the actuator 170 and the gate 140 when the injector 100 is in the non-deployed state while FIG. 31 illustrates the relative positioning of the actuator 170 and the gate 140 during actuation of the actuator 170. As shown in FIGS. 30A and 30B, when the injector 130 is in the non-deployed state, the legs 188A, 188B of the actuator 170 are spaced apart from the gate 140. Depression or downward displacement of the actuator 170 causes the legs 188A, 188B of the actuator 170 to contact and press against the gate 140, as shown in FIG. 31. Particularly, when the actuator 170 is actuated, the legs 188A, 188B of the actuator 170 contact the lateral wings 146A, 146B, respectively, of the gate 140 to push or move the gate 140 downwards in a direction away from the magazine tube mount 134. The gate 140 is thus moved or displaced from the closed configuration in which the gate 140 covers or blocks the distal end or outlet 133 of the magazine tube 132 into the open configuration in which the gate 140 does not cover or block the distal end or outlet 133 of the magazine tube 132, as described above with respect to FIGS. 16A and 16B.

The second stage of actuation is shown in FIGS. 32 and 33. More particularly, FIG. 32 is an enlarged sectional view of the injector 100 that illustrates the relative positioning of the actuator 170 and the shuttle subassembly 150 when the injector 100 is in the non-deployed state, while FIG. 33 illustrates the relative positioning of the actuator 170 and the shuttle subassembly 150 when the injector 100 is in the deployed state. As shown in FIG. 32, when the actuator 170 is in the non-deployed position, the actuator 170 contacts and engages the shuttle body 152 of the shuttle subassembly 150. Stated another way, prior to deployment or operation of the injector 100, the actuator 170 is coupled to the shuttle body 152 and is configured to hold or retain the shuttle body 152 in a first position in which the spring 156 is stretched into the extended configuration. The proximally-extending arms 172A, 172B of the actuator 170 are angled downward such that the proximal end surfaces 174A, 174B thereof contact and abut against the distal end surfaces 155A, 155B, respectively, of the shuttle subassembly 150.

When the actuator 170 is in the deployed position, as shown in FIG. 33, the proximally-extending arms 172A, 172B of the actuator 170 extend approximately parallel to the longitudinal axis of the injector 100. The proximal end surfaces 174A, 174B of the actuator 170 no longer contact or abut against the distal end surfaces 155A, 155B, respectively, of the shuttle subassembly 150. Stated another way, the actuator 170 does not contact the shuttle subassembly 150 and is disengaged or decoupled therefrom. Complete or full depression of the actuator 170 releases the shuttle subassembly 150, thereby permitting the spring 156 to resume to the non-extended or coiled configuration. Coiling of the spring 156 moves or translates the shuttle body 152 and the push rod 160 in a distal direction. Stated another way, the shuttle body 152 and the push rod 160 attached thereto are advanced towards the magazine tube 132 when the spring 156 is permitted to resume to the coiled configuration.

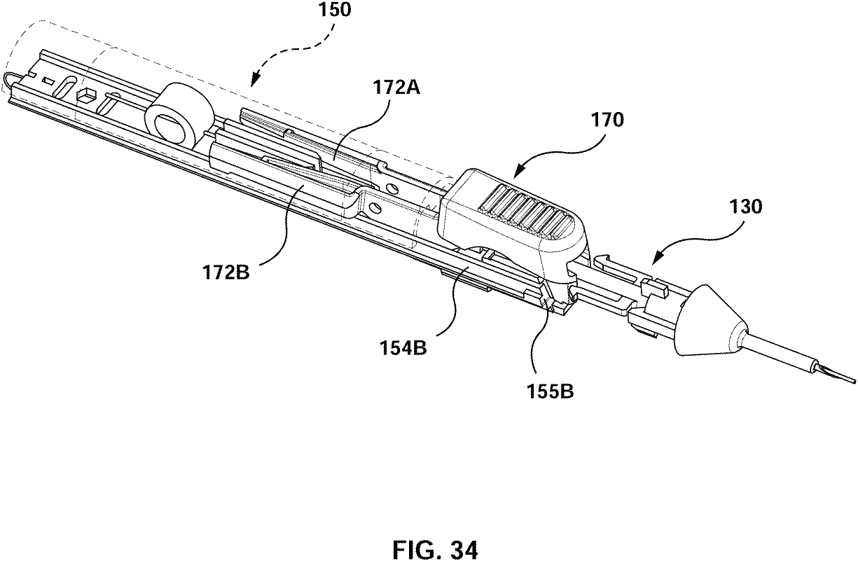

In an embodiment, the actuator 170 is configured to lock out after operation of the injector 100. Stated another way, the actuator 170 cannot be reset to the non-deployed position and may only be depressed once so that the injector 100 is a single-use device. As shown in FIG. 34 in which the injector 100 is shown in the deployed state and the shuttle subassembly 150 is shown in phantom, after deployment of the actuator 170, the proximally-extending arms 172A, 172B of the actuator 170 are disposed within the shuttle subassembly 150. Thus, the proximally-extending arms 172A, 172B cannot be moved or reset to be angled downward due to the placement of the shuttle subassembly 150 relative thereto. Stated another way, after deployment, the shuttle subassembly 150 blocks or prevents the actuator 170 from being reset to the non-deployed position.

In an embodiment, the injector 100 may include one or more components to control or slow down the rate at which the shuttle subassembly 150 moves within the housing 102. When delivering implants into eye tissue, a very controlled release is desirable to avoid damage to the eye. If the implants eject from the injector with too much force, the implants can hit the back of the eye and/or damage the retina of the eye. In an embodiment, the injector 100 is configured to complete delivery, from an initiation to delivery of the implants, within between 3 and 10 seconds.

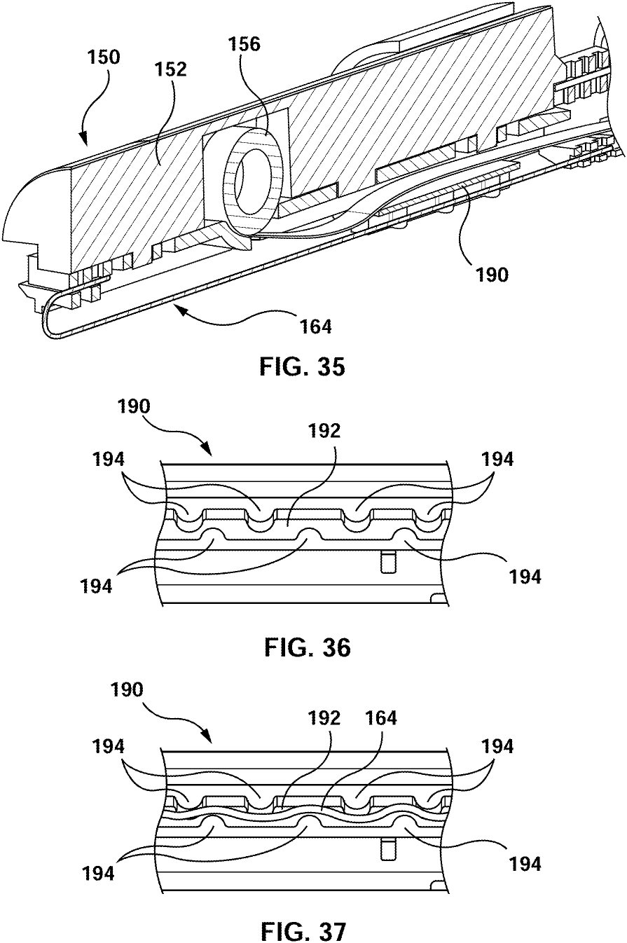

In order to control or slow down the rate at which the shuttle subassembly 150 moves within the housing 102, the injector 100 may include a shuttle decelerator 190 disposed within the housing 102 as shown in FIGS. 35-37. FIG. 35 is an enlarged sectional view of the shuttle subassembly 150 and the shuttle decelerator 190 of the injector 100, while FIG. 36 is a perspective view of the shuttle decelerator removed from the injector 100 for sake of illustration. FIG. 37 is an enlarged perspective view that illustrates the drag wire 164 of the shuttle subassembly 190 as disposed through the shuttle decelerator 190. The shuttle decelerator 190 includes a sinusoidal or wavy groove or path 192. In an embodiment, the sinusoidal path 192 is defined via a plurality of bosses 194. Each boss 194 has a circular or semi-circular profile. The plurality of bosses 194 are spaced apart from each other longitudinally, and are disposed on opposing sidewalls of the path 192 to define the sinusoidal path 192. As best shown in FIG. 37, the drag wire 164 extends through the sinusoidal path 192. Interaction between the drag wire 164 and the plurality of bosses 194 creates friction which slows down or decreases the rate of movement of the shuttle subassembly 150 within the housing 102. In an embodiment, the plurality of bosses 194 are formed from a plastic material and the drag wire 164 is formed from stainless steel. The shuttle decelerator 190 may be secured to an interior surface of the housing 102 and/or a surface of the window chassis 119, as long as it is positioned such that the drag wire 164 passes through the sinusoidal path 192. Although the shuttle decelerator 190 is shown with the sinusoidal path, various shapes or patterns may be utilized to create friction with the drag wire 164.

Although the drag wire 164 and the shuttle decelerator 190 are described above to control or slow down the rate at which the shuttle subassembly 150 moves within the housing 102, other components may be used as an alternative to or in addition to the shuttle decelerator 190. In another embodiment, the injector includes a rotary damper to control or slow down the rate at which the shuttle subassembly moves within the housing 102. Rotary dampers utilize the principle of fluid resistance to dampen movement, and are commercially available via various manufacturers including ACE Controls Inc., of Farmington Hills, Michigan. Specifically, oil viscosity is utilized to provide a braking force of the damper. A damping torque of the rotary damper is determined by the viscosity of the oil, as well as spacing and surface area of the internal components of the rotary damper. In embodiments hereof, as the viscous damping fluid, silicone oil or any other suitable viscous fluid may be used. Silicone oil is commercially available in various viscosity which affects the damping force of the rotary damper. In an embodiment, rotary dampers described herein may utilize methyphenyl silicone fluids or may utilize dimethyl silicone fluids.

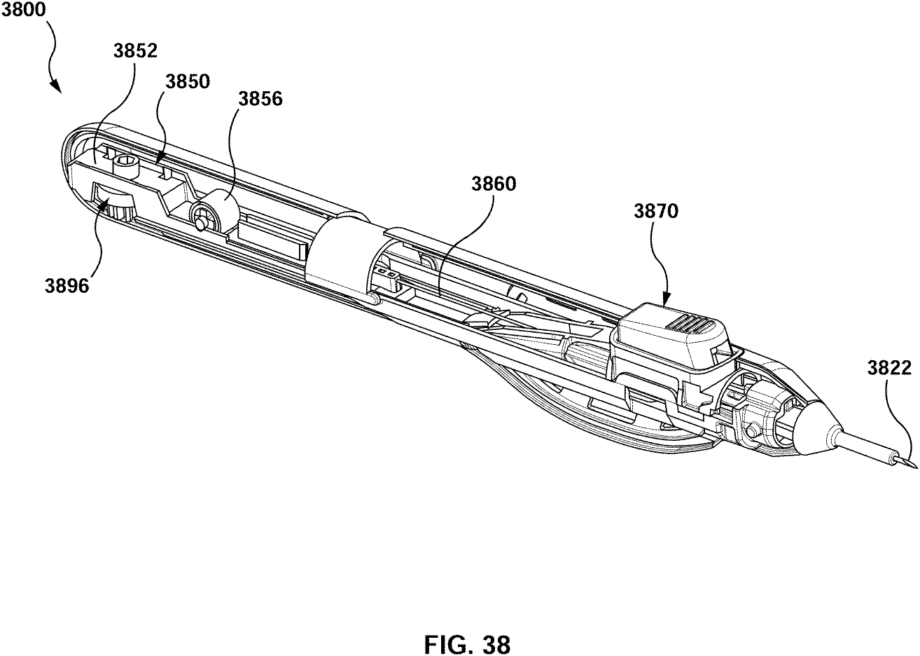

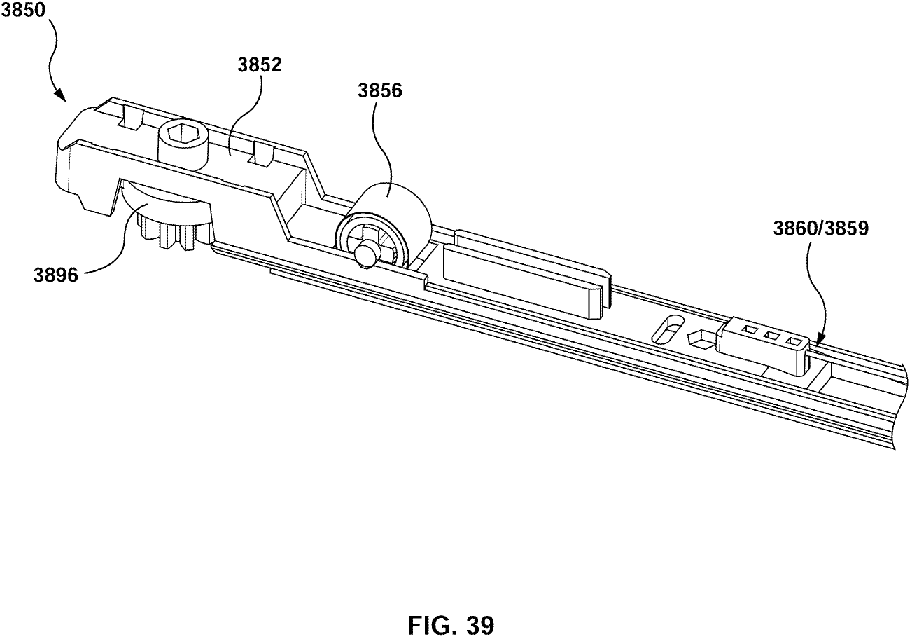

An embodiment of an injector 3800 is shown in FIGS. 38 and 39 which utilizes a rotary damper 3896 to decelerate or dampen movement of a shuttle subassembly 3850 therein. The injector 3800 is the same as the injector 100, except for the differences described herein to the shuttle subassembly. FIG. 39 is a perspective view of the shuttle subassembly 3850 removed from the injector 100 for sake of illustration. The shuttle subassembly 3850 includes a shuttle body 3852, a spring 3856, a pushrod 3860, and the rotary damper 3896. A distal portion of the shuttle body 3852 is configured for interacting with an actuator 3870 of the injector 3800, as described above with respect to the actuator 170 of the injector 100.

The push rod 3860 is the same as the push rod 160 described above. A proximal end 3859 of the push rod 3860 is fixed or secured to the shuttle body 3852 so that the push rod 3860 moves or translates with the shuttle body 3852. When the shuttle body 3852 moves distally, i.e., in a direction towards a cannula 3822, the push rod 3852 is configured to enter the lumens of the magazine tube (not visible in FIG. 38) and the cannula 3822, respectively, to push or distally advance the implants from the magazine tube, into and through the cannula 3822, and ultimately ejected through the outlet or distal end of the cannula 3822 into the eye.

The spring 3856 is the same as the spring 156 described above. The spring 3856 is housed or attached to the shuttle body 3852. The spring 3856 is a constant force spring that is biased or shape-set into a coiled, or non-extended, configuration. Prior to deployment or operation of the injector 3800, as explained above with respect to the injector 100, the actuator 3870 is coupled to the shuttle body 3852 and is configured to hold or retain the shuttle body 3852 in a first position in which the spring 3856 is stretched into the extended configuration. When the actuator 3870 is actuated, the actuator 3870 releases or decouples from the shuttle body 3852 and the spring 3856 is permitted to resume its coiled, or non-extended configuration, due to the biased or shape-set nature thereof. Coiling of the spring 3856 moves or translates the shuttle body 3852 and the push rod 3860 in a distal direction.

The shuttle subassembly 3850 also includes the rotary damper 3896 coupled thereto to control or slow down the rate at which the shuttle subassembly 3850 moves during operation thereof. The rotary damper 3896 is a commercially available through via various manufacturers including ACE Controls Inc., of Farmington Hills, Michigan. In an embodiment, the injector 3800 includes a spring and rotary damper combination configured to obtain a target or desired injection speed. More particularly, the rotary damper is configured to output a particular dampening torque depending on a fluid resistance or viscosity of the fluid within the rotary damper. The injection speed of the injector 3800 is determined by several factors, including the dampening torque of the rotary damper and the spring constant of the spring 3856. In an embodiment, the spring 3856 has a spring constant for load of 0.5 pounds of force (0.5 lbf), the rotary damper has a damping torque of 0.035 in-lbs, and the injector 3800 has an injection speed of between 4 and 9 seconds, or approximately 6.5 seconds with a tolerance of 2.5 seconds. In another embodiment, the spring 3856 has a spring constant for load of 0.5 pounds of force (0.5 lbf), the rotary damper has a damping torque of 0.035 in-lbs, and the injector 3800 has an injection speed of between 5 and 8 seconds, or approximately 6.5 seconds with a tolerance of 1.5 seconds. In another embodiment, the spring 3856 has a spring constant for load of 0.5 pounds of force (0.5 lbf), the rotary damper has a damping torque of 0.035 in-lbs, and the injector 3800 has an injection speed of between 5.5 and 7.5 seconds, or approximately 6.5 seconds with a tolerance of 1 second. In another embodiment, the spring 3856 has a spring constant for load of 0.4 pounds of force (0.4 lbf), the rotary damper has a damping torque of 0.026 in-lbs, and the injector 3800 has an injection speed of between 2.5 and 7.5 seconds, or approximately 5 seconds with a tolerance of 2.5 seconds. In another embodiment, the spring 3856 has a spring constant for load of 0.4 pounds of force (0.4 lbf), the rotary damper has a damping torque of 0.026 in-lbs, and the injector 3800 has an injection speed of between 3.5 and 6.5 seconds, or approximately 5 seconds with a tolerance of 1.5 seconds. In another embodiment, the spring 3856 has a spring constant for load of 0.4 pounds of force (0.4 lbf), the rotary damper has a damping torque of 0.026 in-lbs, and the injector 3800 has an injection speed of between 2.5 and 7.5 seconds, or approximately 5 seconds with a tolerance of 1 second. In another embodiment, the spring 3856 has a spring constant for load of 0.5 pounds of force (0.5 lbf), the rotary damper has a damping torque of 0.026 in-lbs, and the injector 3800 has an injection speed of between 1.5 and 6.5 seconds, or approximately 4 seconds with a tolerance of 2.5 seconds. In another embodiment, the spring 3856 has a spring constant for load of 0.5 pounds of force (0.5 lbf), the rotary damper has a damping torque of 0.026 in-lbs, and the injector 3800 has an injection speed of between 2.5 and 5.5 seconds, or approximately 4 seconds with a tolerance of 1.5 seconds. In another embodiment, the spring 3856 has a spring constant for load of 0.5 pounds of force (0.5 lbf), the rotary damper has a damping torque of 0.026 in-lbs, and the injector 3800 has an injection speed of between 3 and 5 seconds, or approximately 4 seconds with a tolerance of 1 second.

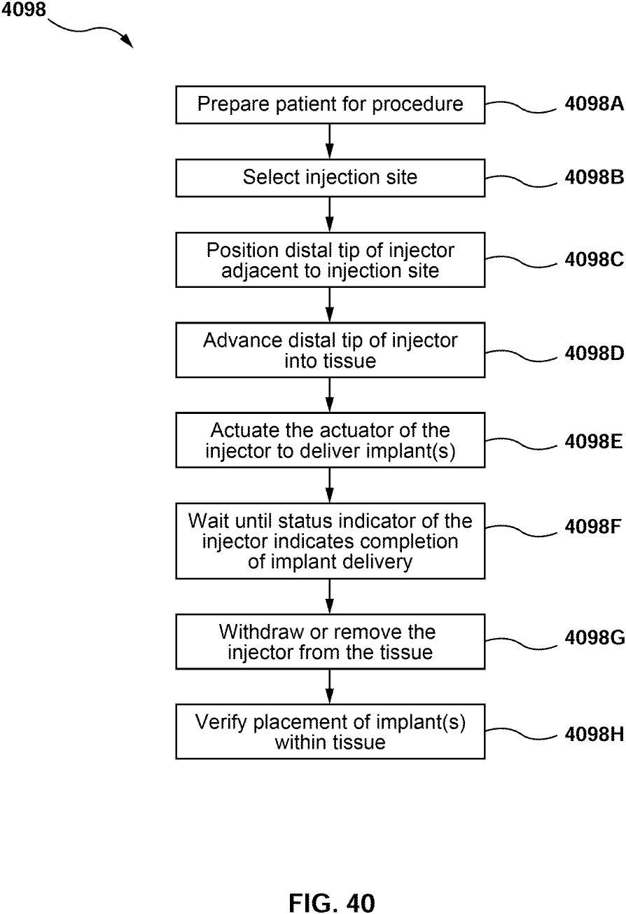

Turning now to FIG. 40, a method 4098 of using an injector according to an embodiment hereof is described in more detail. This method may be used to inject one or more implants 138 into eye tissue, e.g., through the sclera of an eye, with the injector 100 or the injector 3800. For sake of illustration, the method is described herein using the injector 100. In an embodiment, after delivery into the eye tissue, the implants 138 are configured to deliver vorolanib into the vitreous humor for at least 6 months.

In a step 4098A of the method 4098, the patient is prepared for the injection procedure. The patient will typically be under a topical or local anesthetic for an intravitreal injection. Adequate anesthesia and a broad-spectrum microbicide may be given to the patient prior to the injection. The injection procedure should be performed per standard sterile procedures.

In a step 4098B of the method 4098, an injection site is selected or identified and a lid speculum may be positioned on the patient’s eye. In an embodiment, the injection site is between 3.5 mm to 4.0 mm posterior to the limbus in inferior quadrant to ensure optimal safe location for insertion. Once the injection site is selected, the conjunctiva should be gently displaced, using forceps, so that after withdrawing the injector, the conjunctival and scleral needle entry sites will not align. The user may remove the safety cap 104 from the injector 100 such that the injector 100 is ready for injection.

In a step 4098C of the method 4098, the distal end 123 of the cannula 122 is positioned adjacent to or near the injection site, which is the desired point of entry into the tissue. The injector 100 may be mounted on a stand or supported by the hand of a user. The injector 100 may be operated with one hand in a typical clinical environment.

In a step 4098D of the method 4098, the distal end 123 of the cannula 122 is advanced into the tissue to position the cannula 122 at a desired location within the patient’s tissue for deposition of the implants 138. In an embodiment, the distal end 123 of the cannula 122 is advanced until the distal end of the tubular portion 128 of the cannula mount 120 abuts against the outer eye surface. Stated another way, the portion of the cannula 122 which extends distally beyond a distal end of the tubular portion 128 of the cannula mount 120 is intended for insertion into the eye. Thus, the tubular portion 128 of the cannula mount 120 serves as a stop to limit the insertion depth of the distal end 123 of the cannula 122 into the eye tissue.

In an embodiment, the distal end 123 of the cannula 122 is inserted at an oblique angle (i.e., an angle which is not perpendicular or ninety degrees). An oblique insertion angle may promote self-healing of the entry site after the injector 100 is removed. In an embodiment, the cannula 122 may approach the sclera at an approximately 45-degree angle. Once the bevel of the cannula 122 is fully in the sclera, the cannula 122 should be directed toward the mid vitreous and the cannula angle should be directly perpendicular to the sclera. However, an oblique insertion angle is not required and thus in another embodiment, the distal end 123 of the cannula 122 is inserted at a substantially perpendicular angle (i.e., an angle which is approximately ninety degrees).

In a step 4098E of the method, the actuator 170 of the injector 100 is actuated to begin or initiate delivery of the implants 138. More particularly, the user actuates or depresses the actuator 170 to deliver the implants 138 from the initial position within the magazine tube 132 and out of the distal end 123 of the cannula 122. The distal end 123 of the cannula 122 remains inserted in the eye tissue until the status indicator of the injector 100 indicates completion of the injection and thereby verifies that the implants 138 have been successfully delivered out of the distal end 123 of the cannula, as shown in a step 4098F of the method 4098.

In a step 4098G of the method, after the injection is shown to be complete via the status indicator as described above, the injector 100 is withdrawn or removed from the tissue. The user may verify placement of the implants 138 within tissue as shown in a stop 4098H of the method 4098, administer topical antibiotic to the patient, and/or remove the lid speculum from the patient.

It will be understood by one of ordinary skill in the art that certain dimensions or sizes of the components of the injector 100 may vary depending upon the number and size of implants 138 being delivered by the injector. For example, if relatively shorter implants are being delivered and/or only a single implant is being delivered, the push rod 160 and/or shuttle assembly 150 may need to be longer than if relatively longer implants are being delivered. Similarly, the size or gauge of the cannula 122 may vary depending on the type of implant being delivered. Such changes are within the scope of the invention to accommodate delivery of different lengths and numbers of implants by a single injection via the injector 100.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An injector comprising:
a housing;
a push rod disposed at least partially within the housing;
a magazine tube disposed within the housing and having an inlet, an outlet, and a lumen extending from the inlet to the outlet, the magazine tube configured to slidingly receive at least one implant therein, wherein the push rod is configured to be slidingly received within the lumen of the magazine tube;
a gate disposed within the housing, wherein the gate has a closed configuration in which it covers the outlet of the magazine tube and an open configuration in which it does not cover the outlet of the magazine tube;
a cannula having a distal end that is disposed outside of the housing and is configured to be inserted into an eye, wherein a lumen of the cannula is in fluid communication with the lumen of the magazine tube when the gate is in the open configuration; and
an actuator, wherein actuation of the actuator moves the gate from the closed configuration to the open configuration and causes translation of the pushrod through the magazine tube and the cannula.

2. The injector of claim 1, further comprising a safety cap that is configured to be removably coupled to the housing to cover the distal end of the cannula when the safety cap is coupled to the housing, wherein the safety cap includes a tab that extends into a slot formed in the actuator when the safety cap is coupled to the housing to prevent actuation of the actuator.

3. The injector of claim 1, wherein the housing has a generally tubular construction with an asymmetrical fin that includes a height that is greater than a height of the remaining length of the housing.

4. The injector of claim 1, wherein the distal end of the cannula is beveled.

5. The injector of claim 1, wherein the push rod is attached to a shuttle body that is slidingly disposed within the housing and the shuttle body is coupled to a spring, the spring including a non-extended configuration and an extended configuration and being biased to the non-extended configuration.

6. The injector of claim 5, wherein the spring is coiled in the non-extended configuration.

7. The injector of claim 5, wherein the actuator in a non-deployed position holds the shuttle body such that the spring is in the extended configuration.

8. The injector of claim 7, wherein actuation of the actuator from the non-deployed position to a deployed position releases the shuttle body and permits the spring to resume the non-extended configuration.

9. The injector of claim 8, wherein when the actuator is in the non-deployed position, the actuator contacts and engages the shuttle body and when the actuator is in the deployed position, the actuator does not contact the shuttle body and is disengaged therefrom.

10. The injector of claim 8, wherein the actuator is configured to rotate relative to the shuttle body to transition between the non-deployed position and the deployed position.

11. The injector of claim 1, wherein the magazine tube is configured to hold up to three implants and the injector is configured to deliver the three implants via a single actuation of the actuator.

12. The injector of claim 1, wherein the cannula and the magazine tube are coaxially aligned and a transition gap extends between the outlet of the magazine tube and an inlet of the cannula.

13. The injector of claim 12, wherein a portion of the gate is disposed within the transition gap when the gate is in the closed configuration and wherein the portion of the gate is not disposed within the transition gap when the gate is in the open configuration.

14. The injector of claim 1, wherein the housing includes a window formed thereon to permit visual feedback relating to the translation of the pushrod.

15. The injector of claim 14, wherein the push rod is attached to a shuttle body that is slidingly disposed within the housing and an outer surface of the shuttle body includes a status indicator thereon for providing the visual feedback through the window.

16. The injector of claim 1, wherein the push rod is attached to a shuttle body that is slidingly disposed within the housing, and wherein a drag wire is attached to the shuttle body, and wherein the injector further includes a shuttle decelerator disposed within the housing, the shuttle decelerator being configured to receive the drag wire within a sinusoidal path thereof.

17. The injector of claim 16, wherein the sinusoidal path of the shuttle decelerator is defined by a plurality of bosses and interaction between the drag wire and the plurality of bosses creates friction that slows down translation of the shuttle body and push rod attached thereto.

18. The injector of claim 17, wherein the drag wire is formed from stainless steel and the plurality of bosses are formed from a plastic material.

19. The injector of claim 1, wherein the push rod is formed from stainless steel.

20. The injector of claim 1, further comprising the at least one implant.

21. The injector of claim 1, wherein the injector further includes a rotary damper disposed within the housing, the rotary damper being coupled to the push rod and configured to slow down the rate at which the push rod moves within the housing.

22. The injector of claim 21, wherein the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.035 in-lbs, and the injector has an injection speed of between 4 and 9 seconds.

23. The injector of claim 21, wherein the push rod is attached to a shuttle body that is slidingly disposed within the housing and the shuttle body is coupled to a spring, and wherein the spring has a spring constant for load of 0.4 pounds of force (0.4 lbf), the rotary damper has a damping torque of 0.026 in-lbs, and the injector has an injection speed of between 2.5 and 7.5 seconds.

24. The injector of claim 21, wherein the push rod is attached to a shuttle body that is slidingly disposed within the housing and the shuttle body is coupled to a spring, and wherein the spring has a spring constant for load of 0.5 pounds of force (0.005 lbf), the rotary damper has a damping torque of 0.026 in-lbs, and the injector has an injection speed of between 1.5 and 6.5 seconds.

25. A method of using an injector to deliver at least one implant into an eye, the method comprising:
positioning the injector near the eye, the injector including a push rod, a magazine tube having an inlet, an outlet, and a lumen extending from the inlet to the outlet, the magazine tube having the at least one implant disposed therein, a gate in a closed configuration in which it covers the outlet of the magazine tube, a cannula, and an actuator;
inserting a distal end of the cannula into tissue of the eye; and
actuating the actuator to deliver the at least one implant into the tissue of the eye, wherein actuation of the actuator moves the gate from the closed configuration to an open configuration in which the gate does not cover the outlet of the magazine tube and wherein actuation of the actuator also causes translation of the pushrod through the magazine tube and the cannula to push the at least one implant.

26. The method of claim 25, wherein the at least one implant includes exactly three implants.

27. The method of claim 25, wherein the tissue of the eye includes a vitreous of the eye.

28. The method of claim 25, wherein a lumen of the cannula is in fluid communication with the lumen of the magazine tube when the gate is in the open configuration.

29. An injector comprising:
a housing;
a push rod disposed at least partially within the housing;
a magazine tube disposed within the housing and having an inlet, an outlet, and a lumen extending from the inlet to the outlet, the magazine tube configured to slidingly receive at least one implant therein, wherein the push rod is configured to be slidingly received within the lumen of the magazine tube;
a cannula having a distal end that is disposed outside of the housing and is configured to be inserted into an eye; and
an actuator, wherein actuation of the actuator causes translation of the pushrod through the magazine tube and the cannula, wherein the magazine tube is configured to hold at least three implants and the injector is configured to deliver the at least three implants via a single actuation of the actuator, and
wherein a speed of delivery of the at least three implants is controlled such that the speed of delivery is between 2 and 12 seconds.

30. The injector of claim 29, further comprising a gate disposed within the housing, wherein the gate has a closed configuration in which it covers the outlet of the magazine tube and an open configuration in which it does not cover the outlet of the magazine tube,
wherein a lumen of the cannula is in fluid communication with the lumen of the magazine tube when the gate is in the open configuration, and
wherein actuation of the actuator also moves the gate from the closed configuration to the open configuration.

31. The injector of claim 30, wherein the cannula and the magazine tube are coaxially aligned and a transition gap extends between the outlet of the magazine tube and an inlet of the cannula.

32. The injector of claim 31, wherein a portion of the gate is disposed within the transition gap when the gate is in the closed configuration and wherein the portion of the gate is not disposed within the transition gap when the gate is in the open configuration.

33. The injector of claim 29, wherein the speed of delivery of the at least three implants is controlled such that the speed of delivery is between 3 and 10 seconds.

34. The injector of claim 29, wherein the speed of delivery of the at least three implants is controlled such that the speed of delivery is between 5 and 7 seconds.

35. The injector of claim 29, further comprising the at least three implants.

36. The injector of claim 29, wherein the injector further includes a rotary damper disposed within the housing, the rotary damper being coupled to the push rod and configured to slow down the rate at which the push rod moves within the housing.

37. The injector of claim 36, wherein the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.035 in-lbs, and the injector has an injection speed of between 4 and 9 seconds.

38. The injector of claim 36, wherein the push rod is attached to a shuttle body that is slidingly disposed within the housing and the shuttle body is coupled to a spring, and wherein the spring has a spring constant for load of 0.4 pounds of force (0.4 lbf), the rotary damper has a damping torque of 0.026 in-lbs, and the injector has an injection speed of between 2.5 and 7.5 seconds.

39. The injector of claim 36, wherein the push rod is attached to a shuttle body that is slidingly disposed within the housing and the shuttle body is coupled to a spring, and wherein the spring has a spring constant for load of 0.5 pounds of force (0.5 lbf), the rotary damper has a damping torque of 0.026 in-lbs, and the injector has an injection speed of between 1.5 and 6.5 seconds.

40. An injector comprising:
a housing including a window formed thereon;
a push rod disposed at least partially within the housing;
a magazine tube disposed within the housing and having an inlet, an outlet, and a lumen extending from the inlet to the outlet, the magazine tube configured to slidingly receive at least one implant therein, wherein the push rod is configured to be slidingly received within the lumen of the magazine tube;
a cannula having a distal end that is disposed outside of the housing and is configured to be inserted into an eye; and
an actuator, wherein actuation of the actuator causes translation of the pushrod through the magazine tube and the cannula,
wherein the window permits visual feedback of translation of the pushrod through the housing, the visual feedback providing an indication that delivery of the at least one implant is complete.

41. The injector of claim 40, further comprising a gate disposed within the housing, wherein the gate has a closed configuration in which it covers the outlet of the magazine tube and an open configuration in which it does not cover the outlet of the magazine tube,
wherein a lumen of the cannula is in fluid communication with the lumen of the magazine tube when the gate is in the open configuration, and
wherein actuation of the actuator also moves the gate from the closed configuration to the open configuration.

42. The injector of claim 41, wherein the cannula and the magazine tube are coaxially aligned and a transition gap extends between the outlet of the magazine tube and an inlet of the cannula.

43. The injector of claim 42, wherein a portion of the gate is disposed within the transition gap when the gate is in the closed configuration and wherein the portion of the gate is not disposed within the transition gap when the gate is in the open configuration.

44. The injector of claim 40, wherein the push rod is attached to a shuttle body that is slidingly disposed within the housing and an outer surface of the shuttle body includes at least one status indicator thereon for providing the visual feedback through the window.

45. The injector of claim 44, wherein the at least one status indicator includes a first status indicator and a second status indicator, the first status indicator being disposed proximal to the second status indicator, and wherein the first status indicator is displayed through the window prior to actuation of the actuator and the second status indicator is displayed through the window when the delivery of the at least one implant is complete.

46. The injector of claim 45, wherein the at least one status indicator further includes a third status indicator disposed between the first status indicator and the second status indicator, and wherein the second status indicator is disposed through the window while the shuttle body is moving within the housing.

47. The injector of claim 46, wherein the first status indicator is a first color, the second status indicator is a second color, and the third status indicator is a third color.

48. The injector of claim 40, further comprising the at least one implant.

49. A method of using an injector to deliver at least one implant into an eye, the method comprising:
positioning a distal tip of the injector adjacent to an injection site of the eye, the injector including a push rod, a magazine tube having an inlet, an outlet, and a lumen extending from the inlet to the outlet, the magazine tube having the at least one implant disposed therein, a gate in a closed configuration in which it covers the outlet of the magazine tube, a cannula, an actuator; and a status indicator;
advancing the distal end of the injector into tissue of the eye at the injection site;
actuating the actuator to deliver the at least one implant into the tissue of the eye, wherein actuation of the actuator moves the gate from the closed configuration to an open configuration in which the gate does not cover the outlet of the magazine tube and wherein actuation of the actuator also causes translation of the pushrod through the magazine tube and the cannula to push the at least one implant;
maintaining the position of the distal end of the injector within the tissue of the eye until the status indicator of the injector indicates completion of implant delivery; and
removing the injector from the tissue of the eye after the status indicator of the injector indicates completion of implant delivery.

50. The method of claim 49, wherein the at least one implant includes exactly three implants.

51. The method of claim 49, wherein the at least one implant is used to treat chronic non-infectious uveitis affecting the posterior segment of the eye in an eye in need thereof.

52. The method of claim 49, wherein the tissue of the eye includes a vitreous of the eye.

53. The method of claim 49, wherein the implant is an intravitreal implant comprising about 0.18 mg fluocinolone acetonide.

54. The method of claim 53, wherein the implant also comprises polyvinyl alcohol, silicone adhesive, a polyimide tube and may comprise water.

55. The method of claim 49, wherein the at least one implant is used to treat a retinal disease in an eye in need thereof.

56. The method of claim 55, wherein the retinal disease is selected from wet AMD, diabetic retinopathy, diabetic macular edema and retinal vein occlusion.

57. The method of claim 55, wherein the implant is an intravitreal implant comprises about 400 µg to about 2800 µg vorolanib.

58. The method of claim 57, wherein the implant also comprises polyvinyl alcohol.
